SEQUENCE LISTING (61 sequences)

SEQ ID NO: 1 - protein, 453 aa, Homo sapiens [sequence omitted]

SEQ ID NO: 2 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 3 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 5 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 6 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 7 - protein, 121 aa, Artificial Sequence [sequence omitted]

SEQ ID NO: 8 - protein, 107 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 9 - protein, 107 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSDPPTFGGGTKLEIKR

SEQ ID NO: 10 - protein, 107 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSEPPTFGGGTKLEIKR

SEQ ID NO: 11 - protein, 107 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSAPPTFGGGTKLEIKR

SEQ ID NO: 12 - protein, 107 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSKPPTFGGGTKLEIKR

SEQ ID NO: 13 - protein, 107 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSRPPTFGGGTKLEIKR

SEQ ID NO: 14 - protein, 122 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 15 - protein, 112 aa, Artificial Sequence
  Description of Artificial Sequence Synthetic polypeptide
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 16 - protein, 118 aa, Artificial Sequence
  Description of Artificial Sequence Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 17 - protein, 108 aa, Artificial Sequence
  Description of Artificial Sequence Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 18 - protein, 125 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 19 - protein, 107 aa, Artificial Sequence
  Description of Artificial Sequence Synthetic polypeptide
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 20 - protein, 120 aa, Artificial Sequence [sequence omitted]

SEQ ID NO: 21 - protein, 107 aa, Artificial Sequence [sequence omitted]

SEQ ID NO: 22 - protein, 119 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 23 - protein, 107 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 24 - protein, 122 aa, Artificial Sequence [sequence omitted]

SEQ ID NO: 25 - protein, 106 aa, Artificial Sequence [sequence omitted]

SEQ ID NO: 26 - protein, 119 aa, Artificial Sequence
  Description of Artificial Sequence Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 27 - protein, 115 aa, Artificial Sequence
  Description of Artificial Sequence Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 28 - protein, 119 aa, Artificial Sequence [sequence omitted]

SEQ ID NO: 29 - protein, 107 aa, Artificial Sequence [sequence omitted]

SEQ ID NO: 30 - protein, 121 aa, Artificial Sequence
  Description of Artificial Sequence Synthetic polypeptide
EVQLVESGGGLVQPGGSLRLSCAVSGYSITSGYSWNWIRQAPGKGLEWVASITYDGSTNYADSVKGRFTISRDDSKNTFYLQMNSLRAEDTAVYYCARGSHYFGHWHFAVWGQGTLVTVSS

SEQ ID NO: 31 - protein, 111 aa, Artificial Sequence [sequence omitted]

SEQ ID NO: 32 - protein, 120 aa, Artificial Sequence [sequence omitted]

SEQ ID NO: 33 - protein, 111 aa, Artificial Sequence [sequence omitted]

SEQ ID NO: 34 - protein, 120 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 35 - protein, 110 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 36 - protein, 118 aa, Artificial Sequence [sequence omitted]

SEQ ID NO: 37 - protein, 107 aa, Artificial Sequence [sequence omitted]

SEQ ID NO: 38 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 39 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 40 - protein, 113 aa, Artificial Sequence [sequence omitted]

SEQ ID NO: 41 - protein, 107 aa, Artificial Sequence [sequence omitted]

SEQ ID NO: 42 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 43 - protein, 137 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 44 - protein, 120 aa, Artificial Sequence [sequence omitted]

SEQ ID NO: 45 - protein, 106 aa, Artificial Sequence
  Description of Artificial Sequence Synthetic polypeptide
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIK

SEQ ID NO: 46 - protein, 412 aa, Artificial Sequence
  Description of Artificial Sequence Synthetic polypeptide
MGWSYIILFLVATATGVHSQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 47 - protein, 412 aa, Artificial Sequence
  Description of Artificial Sequence Synthetic polypeptide
MGWSYIILFLVATATGVHSQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 48 - protein, 125 aa, Artificial Sequence
  Description of Artificial Sequence Synthetic polypeptide
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 49 - protein, 10 aa, Artificial Sequence
  Description of Artificial Sequence Synthetic peptide
GFTFNTYAMN

SEQ ID NO: 50 - protein, 20 aa, Artificial Sequence
  Description of Artificial Sequence Synthetic peptide
ARIRSKYNNYATYYADSVKD

SEQ ID NO: 51 - protein, 16 aa, Artificial Sequence
  Description of Artificial Sequence Synthetic peptide
VRHGNFGNSYVSWFAY

SEQ ID NO: 52 - protein, 109 aa, Artificial Sequence
  Description of Artificial Sequence Synthetic polypeptide
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 53 - protein, 14 aa, Artificial Sequence
  Description of Artificial Sequence Synthetic peptide
RSSTGAVTTSNYAN

SEQ ID NO: 54 - protein, 7 aa, Artificial Sequence
  Description of Artificial Sequence Synthetic peptide
GTNKRAP

SEQ ID NO: 55 - protein, 7 aa, Artificial Sequence
  Description of Artificial Sequence Synthetic peptide
ALWYSNL

SEQ ID NO: 56 - protein, 420 aa, Artificial Sequence
  Description of Artificial Sequence Synthetic polypeptide
MGWSYIILFLVATATGVHSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 57 - protein, 5 aa, Artificial Sequence
  Description of Artificial Sequence Synthetic peptide
GGGGS

SEQ ID NO: 58 - protein, 10 aa, Artificial Sequence
  Description of Artificial Sequence Synthetic peptide
GGGGSGGGGS

SEQ ID NO: 59 - protein, 15 aa, Artificial Sequence
  Description of Artificial Sequence Synthetic peptide
GGGGSGGGGSGGGGS

SEQ ID NO: 60 - protein, 20 aa, Artificial Sequence
  Description of Artificial Sequence Synthetic peptide
GGGGSGGGGSGGGGSGGGGS

SEQ ID NO: 61 - protein, 25 aa, Artificial Sequence
  Description of Artificial Sequence Synthetic peptide
GGGGSGGGGSGGGGSGGGGSGGGGS

(19) United States
(12) Patent Application Publication
(10) Pub. No.: US 2022/0372142 A1
(43) Pub. Date: Nov. 24, 2022

(54) MULTIMERIC ANTIBODIES WITH ENHANCED SELECTIVITY FOR CELLS WITH HIGH TARGET DENSITY

(71) Applicant: IGM Biosciences, Inc., Mountain View, CA (US)

(72) Inventors: Ramesh BALIGA, Redwood City, CA (US); Paul HINTON, Sunnyvale, CA (US); Bruce KEYT, Hillsborough, CA (US)

(21) Appl. No.: 17/761,428

(22) PCT Filed: Sep. 18, 2020
(86) PCT No.: PCT/US2020/051513

§ 371 Date: Mar. 17, 2022

Related U.S. Application Data
(60) Provisional application No. 62/902,915, filed on Sep. 19, 2019.

(51) Int. Cl. C07K 16/28; C07K 14/54; C07K 14/715; A61P 35/00

(52) U.S. Cl. CPC C07K 16/283; C07K 14/5443; C07K 14/7155; C07K 16/2809; A61P 35/00; C07K 16/2887; C07K 2317/35; C07K 2317/622; C07K 2319/30; C07K 2317/92; C07K 2317/734; C07K 2317/31

(57) ABSTRACT

This disclosure provides a multimeric binding molecule, e.g., an IgM, IgM-like, IgA, or IgA-like binding molecule, e.g., an antibody with enhanced selectivity for cells expressing a target antigen at high density, e.g., tumor cells. Enhanced selectivity can be achieved, e.g., through modulation of binding affinity for the target antigen and/or through adjusting avidity via multimeric antigen binding.

### CROSS-REFERENCE TO RELATED APPLICATIONS

This application claims the benefit of U.S. Provisional Patent Application Ser. No. 62/902,915, filed Sep. 19, 2019, which is incorporated herein by reference in its entirety.

### SEQUENCE LISTING

The instant application contains a Sequence Listing which has been submitted electronically in ASCII format and is hereby incorporated by reference in its entirety. The ASCII copy was created on Sep. 17, 2020, is named 023WO1-Sequence-Listing, and is 82,283 bytes in size.

### BACKGROUND

Monoclonal antibodies that directly target cancer cells and other diseased cells typically bind to target antigens that are overexpressed on the diseased cells but are also expressed at a reduced level on normal, healthy cells. Accordingly, while treatment with such an antibody can kill tumor cells, the antibody can also have an adverse effect on normal cells. For example, the anti-CD20 monoclonal antibody rituximab can effectively kill B-cell lymphomas but can also deplete the patient's normal B-cells. Kosmas, C., et al., Leukemia 16:2004-2015 (2002). Recently, Slaga et al. Science Trans. Med. doi:10.1126/scitranslmed.aat5775 (2018) demonstrated that bispecific anti-HER2/CD3 antibodies with bivalent binding to HER2 but with reduced affinity showed selective killing of HER2-overexpressing tumor cells relative to killing of non-tumor cells expressing a lower density of HER2.

Antibodies and antibody-like molecules that can multimerize, such as IgA and IgM antibodies, have emerged as promising drug candidates in the fields of, e.g., immuno-oncology and infectious diseases allowing for improved specificity and avidity, and also the ability to bind to multiple binding targets. See, e.g., U.S. Pat. Nos. 9,951,134, 9,938,347, and 10,618,978, U.S. Patent Application Publication Nos. US 2019-0100597 and US 2019-0185570, and PCT Publication Nos. WO 2016/154593, WO 2016/168758, WO 2018/017888, WO 2018/017763, WO 2018/017889, WO 2018/017761, and WO 2019/169314, the contents of which are incorporated herein by reference in their entireties.

There remains a need for therapeutic monoclonal antibodies that can more selectively target tumor or other diseased cells that overexpress the target antigen, while avoiding interactions with healthy cells that express the antigen at reduced density.

### SUMMARY

This disclosure provides a multimeric binding molecule that includes two, five, or six bivalent binding units, that collectively include four, ten, or twelve binding unit-associated antigen binding domains, respectively. As provided herein each binding unit includes two antibody heavy chains, each having an IgA, IgA-like, IgM, or IgM-like heavy chain constant region or multimerizing fragment or variant thereof and a binding unit-associated antigen-binding domain or subunit thereof, where at least three of the binding-unit-associated antigen-binding domains of the binding molecule specifically bind to the same predetermined target on the surface of a cell, and where the binding molecule preferentially binds to a cell expressing the predetermined target at a higher density relative to a cell expressing the predetermined target at a lower density.

In certain embodiments, each antibody heavy chain includes a heavy chain variable region (VH) portion of a binding-unit-associated antigen-binding domain. In certain embodiments, at least three binding unit-associated antigen-binding domains that bind to the same predetermined target on the surface of a cell each include a heavy chain variable region (VH) and light chain variable region (VL). In certain embodiments at least three antigen-binding domains each bind to the same epitope on the predetermined target. In certain embodiments the at least three binding-unit-associated antigen-binding domains that bind to the same predetermined target on the surface of a cell are identical.

In certain embodiments at least four, at least five, at least six, at least seven, at least eight, at least nine, at least ten, at least eleven, or twelve binding-unit-associated antigen-binding domains bind to the same predetermined target on the surface of a cell and are identical. In certain embodiments, the provided binding molecule preferentially binds to a cell expressing the predetermined target at a higher density relative to a reference bivalent IgG antibody having just two of the identical binding-unit-associated antigen-binding domains that specifically bind to the predetermined target on the surface of the cell. In certain embodiments, the provided binding molecule can bind to a cell expressing the target antigen at higher density, where the reference bivalent IgG antibody having just two of the identical binding-unit-associated antigen-binding domains that specifically bind to the predetermined target on the surface of the cell cannot bind to the cell expressing the target antigen at higher density. In certain embodiments the provided binding molecule does not detectably bind to a cell expressing the predetermined target at lower density. In certain embodiments the cell expressing the predetermined target at high density is a cancer cell and the cell expressing the predetermined target at low density is a normal, healthy cell.

In certain embodiments the multimeric binding molecule is pentameric or hexameric, and includes five or six bivalent IgM or IgM-like binding units, respectively, where each binding unit includes two IgM or IgM-like heavy chain constant regions or multimerizing fragments or variants thereof each associated with a binding unit-associated antigen-binding domain or subunit thereof. In certain embodiments the IgM or IgM-like heavy chain constant regions or multimerizing fragments or variants thereof each include a Cμ4 domain and an IgM tailpiece (tp) domain, and can further include a Cp domain, a Cμ2 domain, a Cμ3 domain, or any combination thereof. In certain embodiments the IgM or IgM-like heavy chain constant regions are human IgM constant regions or human-derived IgM-like constant regions. In certain embodiments, each binding unit includes two IgM or IgM-like heavy chains each including a VH situated amino terminal to the IgM or IgM-like constant region or multimerizing fragment or variant thereof, and two immunoglobulin light chains each including a VL situated amino terminal to an immunoglobulin light chain constant region. In those embodiments where the multimeric binding molecule is pentameric, it can further include a J-chain, or functional fragment or variant thereof.

In certain embodiments the multimeric binding molecule is dimeric and includes two bivalent IgA or IgA-like binding units and a J-chain or a functional fragment or variant thereof, where each binding unit includes two IgA or IgA-like heavy chain constant regions or multimerizing fragments or variants thereof each associated with a binding unit-associated antigen-binding domain or subunit thereof. In certain embodiments the dimeric binding molecule can further include a secretory component, or functional fragment or variant thereof. In certain embodiments the IgA or IgA-like heavy chain constant regions or multimerizing fragments or variants thereof each include a Cα3 domain and an IgA tailpiece (tp) domain and can further include a Cal domain, a Cα2 domain, an IgA hinge region, or any combination thereof. In certain embodiments the IgA or IgA-like heavy chain constant regions or multimerizing fragments or variants thereof are human IgA heavy chain constant regions or human-derived IgA-like heavy chain constant regions. In certain embodiments each binding unit includes two IgA or IgA-like heavy chains each including a VH situated amino terminal to the IgA or IgA-like heavy chain constant region or multimerizing fragment or variant thereof, and two immunoglobulin light chains each including a VL situated amino terminal to an immunoglobulin light chain constant region.

Certain multimeric binding molecules provided herein include a J-chain or fragment or variant thereof. In certain embodiments the J-chain or fragment or variant thereof is a human or human-derived J-chain that includes the amino acid sequence SEQ ID NO: 42 or a functional fragment or variant thereof. In certain embodiments the J-chain or functional fragment or variant thereof is a variant J-chain including one or more single amino acid substitutions, deletions, or insertions relative to a wild-type J-chain, which can affect serum half-life of the binding molecule. According to these embodiments, a binding molecule that includes the variant J-chain exhibits an increased serum half-life upon administration to an animal relative to a reference binding molecule that is identical except for the one or more single amino acid substitutions, deletions, or insertions, and is administered in the same way to the same animal species. More specifically, in certain embodiments the J-chain or functional fragment or variant thereof includes an amino acid substitution at the amino acid position corresponding to amino acid Y102 of the wild-type human J-chain (SEQ ID NO: 42). Y102 of SEQ ID NO: 42 can be substituted with alanine (A), serine (S), or arginine (R). More specifically the amino acid corresponding to Y102 of SEQ ID NO: 42 can be substituted with alanine (A). In certain specific embodiments the variant J-chain is a variant human J-chain and includes the amino acid sequence SEQ ID NO: 43 (J*).

In certain embodiments, the J-chain or fragment or variant thereof of a provided multimeric binding molecule is a modified J-chain and further includes a heterologous moiety, where the heterologous moiety is chemically conjugated or fused to the J-chain or fragment or variant thereof. In certain embodiments the heterologous moiety is a heterologous polypeptide. The heterologous polypeptide can be fused to the J-chain or fragment or variant thereof via a peptide linker. For example the peptide linker can consist of the amino acid sequence SEQ ID NO: 57, SEQ ID NO: 58, SEQ ID NO: 59, SEQ ID NO: 60, or SEQ ID NO: 61. In certain embodiments the heterologous polypeptide is fused to the N-terminus of the J-chain or fragment or variant thereof, to the C-terminus of the J-chain or fragment or variant thereof, or heterologous polypeptides can be fused to both the N- and C-terminus of the J-chain or fragment or variant thereof. In certain embodiments at least two heterologous polypeptides can be fused to the J-chain or fragment or variant thereof, and the at least two heterologous polypeptides can be the same or different. In certain embodiments at least one heterologous polypeptide includes a J-chain-associated antigen-binding domain. In certain embodiments the J-chain-associated antigen-binding domain is an antibody or antigen-binding fragment thereof, e.g., an Fab fragment, an Fab′ fragment, an F(ab′)2 fragment, an Fd fragment, an Fv fragment, a single-chain Fv (scFv) fragment, a disulfide-linked Fv (sdFv) fragment, or any combination thereof. In certain embodiments the J-chain-associated antigen-binding domain is a scFv fragment. In certain embodiments the J-chain-associated antigen-binding domain specifically binds to an immune effector cell, e.g., a T cell, e.g., CD8+ cytotoxic T cell, or an NK cell.

Where the immune effector cell is a T cell, the J-chain-associated antigen-binding domain can be, e.g., a scFv fragment that specifically binds to CD3ε. In certain embodiments the anti-CD3ε scFv fragment can include a heavy chain variable region (VH) and a light chain variable region (VL), where the VH includes the VH complementarity-determining regions VHCDR1, VHCDR2, and VHCDR3 including the amino acid sequences SEQ ID NO: 49, SEQ ID NO: 50, and SEQ ID NO: 51, respectively, or SEQ ID NO: 49, SEQ ID NO: 50, and SEQ ID NO: 51 with one, two, or three amino acid substitutions in one or more of the VHCDRs, and where the VL includes the VL complementarity-determining regions VLCDR1, VLCDR2, and VLCDR3 including the amino acid sequences SEQ ID NO: 53, SEQ ID NO: 54, and SEQ ID NO: 55, respectively, or SEQ ID NO: 53, SEQ ID NO: 54, and SEQ ID NO: 55 with one, two, or three amino acid substitutions in one or more of the VLCDRs. For example the scFv fragment can include the VH amino acid sequence SEQ ID NO: 48 and the VL amino a heavy chain variable region (VH) and a light chain variable region (VL), where the VH and VL include the amino acid sequences SEQ ID NO: 44 and SEQ ID NO: 45, respectively. In certain specific embodiments, the modified J-chain can include amino acids 20 to 412 of SEQ ID NO: 46 (V15J), amino acids 20 to 412 of SEQ ID NO: 47 (V15J*), or amino acids 20 to 420 of SEQ ID NO: 56 (SJ*). Where the immune effector cell is an NK cell, the J-chain-associated antigen-binding domain can be, e.g., a scFv fragment that specifically binds to CD16.

In certain embodiments, a modified J-chain of a provided multimeric binding molecule can include an immune stimulatory agent (“ISA”) fused or chemically conjugated to the J-chain or fragment or variant thereof. In certain embodiments ISA can include a cytokine or receptor-binding fragment or variant thereof. For example the IS can include (a) an interleukin-15 (IL-15) protein or receptor-binding fragment or variant thereof (“I”), and (b) an interleukin-15 receptor-α (IL-15Rα) fragment including the sushi domain or a variant thereof capable of associating with I (“R”), where the J-chain or fragment or variant thereof and at least one of I and R are associated as a fusion protein, and where I and R can associate to function as the ISA. In certain embodiments the ISA can be fused to the J-chain via a peptide linker.

In certain embodiments, the predetermined target of the provided multimeric binding molecule is B-cell maturation antigen (BCMA), CD19, CD20, EGFR, HER2 (ErbB2), ErbB3, ErbB4, CTLA4, PD-1, PD-L1, VEGF, VEGFR1, VEGFR2, CD52, CD30, prostate-specific membrane antigen (PSMA), CD38, GD2, SLAMF7, platelet-derived growth factor receptor A (PDGFRA), CD22, FLT3 (CD135), CD123, MUC-16, carcinoembryonic antigen-related cell adhesion molecule 1 (CEACAM-1), mesothelin, tumor-associated calcium signal transducer 2 (Trop-2), glypican-3 (GPC-3), human blood group H type 1 trisaccharide (Globo-H), sialyl Tn antigen (STn antigen), or CD33. In certain embodiments, at least three identical binding unit-associated antigen-binding domains that are specific for the predetermined target are reduced-affinity variants of an antigen-binding domain of an existing antibody known to bind to the predetermined target, where the antigen-binding domain of an existing antibody includes a heavy chain variable region (VH) and light chain variable region (VL). In certain embodiments the existing antibody is alemtuzumab, atezolizumab, atezolizumab, avelumab, bevacizumab, blinatumomab, brentuximab, capromab, cetuximab, daratumumab, denosumab, dinutuximab, durvalumab, elotuzumab, gemtuzumab, ibritumomab, ipilimumab, inotuzumab, necitumumab, nivolumab, nivolumab, obinutuzumab, ocrelizumab, ofatumumab, olaratumab, omalizumab, panitumumab, pembrolizumab, pertuzumab, ramucirumab, ranibizumab, rituximab, trastuzumab, or tremelimumab. In certain embodiments the provided binding molecule binds to the predetermined target with a binding affinity for the predetermined target at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 500-fold, or at least 1000-fold lower than the existing antibody. In certain embodiments the VH and VL of the antigen-binding domain of the existing antibody include, respectively, the amino acid sequences SEQ ID NO: 7 and SEQ ID NO: 8, SEQ ID NO: 14 and SEQ ID NO: 15, SEQ ID NO: 16 and SEQ ID NO: 17, SEQ ID NO: 18 and SEQ ID NO: 19, SEQ ID NO: 20 and SEQ ID NO: 21, SEQ ID NO: 22 and SEQ ID NO: 23, SEQ ID NO: 24 and SEQ ID NO: 25, SEQ ID NO: 26 and SEQ ID NO: 27, SEQ ID NO: 28 and SEQ ID NO: 29, SEQ ID NO: 30 and SEQ ID NO: 31, SEQ ID NO: 32 and SEQ ID NO: 33, SEQ ID NO: 34 and SEQ ID NO: 35, SEQ ID NO: 36 and SEQ ID NO: 37, SEQ ID NO: 38 and SEQ ID NO: 39, or SEQ ID NO: 40 and SEQ ID NO: 41.

More specifically, the antigen-binding domain of the existing antibody specifically binds to CD20, and includes the VH amino acid sequence SEQ ID NO: 7 and the VL amino acid sequence SEQ ID NO: 8, and the reduced-affinity variant includes an amino acid substitution at position N93 in the VL, SEQ ID NO: 8. For example N93 of SEQ ID NO: 8 can include a N93D or N93E amino acid substitution. In certain specific embodiments the at least three identical binding unit-associated antigen-binding domains specific for the predetermined target include the VH amino acid sequence SEQ ID NO: 7 and the VL amino acid sequence SEQ ID NO: 9 or SEQ ID NO: 10. In certain embodiments, the provided binding molecule can direct complement-directed cytotoxicity (CDC) of B cells expressing CD20 at high density at an EC50 concentration at least 2-fold, at least 5-fold, at least 10-fold, at least 50-fold, at least 100-fold, at least 500-fold or at least 1000-fold lower than the EC50 concentration for B cells expressing CD20 at low density. In certain embodiments the provided binding molecule can direct CDC of B cells expressing CD20 at high density, but not B cells expressing CD20 at low density. In certain embodiments the provided anti-CD20 binding molecule is a pentameric IgM or IgM-like binding molecule or a dimeric IgA or IgA-like binding molecule, and further includes a modified J-chain including a scFv that specifically binds to CD3ε. For example, the modified J-chain can include amino acids 20 to 412 of SEQ ID NO: 46 (V15J), amino acids 20 to 412 of SEQ ID NO: 47 (V15J*), or amino acids 20 to 420 of SEQ ID NO: 56 (SJ*). In certain embodiments the provided anti-CD20 binding molecule can direct T-cell directed cellular cytotoxicity (TDCC) or both TDCC and CDC of B cells expressing CD20 at high density at an EC50 concentration at least 2-fold, at least 5-fold, at least 10-fold, at least 50-fold, at least 100-fold, at least 500-fold or at least 1000-fold lower than the EC50 concentration for a B cell line or normal B cells, expressing CD20 at low density. In certain embodiments the provided anti-CD20 binding molecule can direct TDCC or both TDCC and CDC of B cells expressing CD20 at high density, but not B cells expressing CD20 at low density. In certain embodiments the B cells expressing CD20 at both high and low density are lymphoma cell lines. For example, the lymphoma cell line expressing CD20 at high density is a Ramos cell line, a Raji cell line, a DoHH-2 cell line, a JeKo-1 cell line, a Z-138 cell line, a Daudi cell line, a Granta cell line, or a DoHH2 cell line, and the lymphoma cell line expressing CD20 at low density is a CA46 cell line, a Nalm-1 cell line, a Toledo cell line, a BJAB cell line, a Kasumi-2 cell line, an RPMI 8226 cell line, an HT cell line, an SU-DHL-8 cell line, a JM1 cell line, a Namalwa cell line, a Nalm-6 cell line, or a Z138 cell line. In certain embodiments the provided anti-CD20 binding molecule can direct CDC of Ramos cells, where an equivalent amount of a monospecific bivalent IgG1 antibody including the same antigen-binding domains as the binding molecule shows no detectable complement-mediated killing of Ramos cells. In certain embodiments the B cells expressing CD20 at high density are CD20-positive malignant B cells in a subject with cancer, and the B cells expressing CD20 at low density are normal B cells in the subject with cancer. In certain embodiments the cancer is a CD20-positive leukemia, lymphoma, or myeloma. In certain embodiments, the subject is human.

The disclosure further provides a composition that includes the binding molecule provided by the disclosure.

The disclosure further provides an isolated polynucleotide that includes a nucleic acid molecule that encodes the provided binding molecule, an antigen-binding and multimerizing fragment of the provided binding molecule, or a subunit thereof. In certain embodiments the subunit is an antibody heavy chain or multimerizing fragment or variant thereof, an antibody light chain or fragment thereof, a J-chain or fragment or variant thereof, or any combination thereof. The disclosure further provides a vector that includes the provided polynucleotide, and a host cell that includes the provided polynucleotide or the provided vector. In a related embodiment, the disclosure provides a method for producing the provided binding molecule, where the method includes culturing the provided host cell and recovering the binding molecule.

In addition, the disclosure provides an IgM or IgM-like antibody that includes five bivalent binding units and a modified J-chain, where each binding unit includes two human IgM or human-derived IgM-like heavy chain constant regions or multimerizing fragments or variants thereof, each associated with a binding unit-associated antigen-binding domain, where each binding unit-associated antigen-binding domain of the antibody includes the VH amino acid sequence SEQ ID NO: 7 and the VL amino acid sequence SEQ ID NO: 9 or SEQ ID NO: 10, where the modified J-chain includes a J-chain-associated binding domain including a scFv that specifically binds to CD3a, and where the IgM antibody can direct TDCC, CDC, or both TDCC and CDC of B cells expressing CD20 at high density at an EC50 concentration at least 2-fold, at least 5-fold, at least 10-fold, at least 50-fold, at least 100-fold, at least 500-fold or at least 1000-fold lower than the EC50 concentration for a B cell line or normal B cells, expressing CD20 at low density. In certain embodiments, the IgM or IgM-like antibody can direct CDC, TDCC, or both TDCC and CDC of B cells expressing CD20 at high density, but not B cells expressing CD20 at low density. In certain embodiments, the modified J-chain includes amino acids 20 to 412 of SEQ ID NO: 46 (V15J), amino acids 20 to 412 of SEQ ID NO: 47 (V15J*), or amino acids 20 to 420 of SEQ ID NO: 56 (SJ*). In certain aspects, the high and low density CD20-expressing cells are lymphoma cell lines, for example, the cell expressing CD20 at high density can be a Ramos cell line, and the cell expressing CD20 at low density can be a CA46 cell line. In certain embodiments the provided IgM or IgM-like antibody can direct complement-mediated killing of Ramos cells, where an equivalent amount of a monospecific bivalent IgG1 antibody including the same antigen-binding domains shows no detectable complement-mediated killing of Ramos cells. In certain embodiments the cell expressing CD20 at high density is a malignant B cell in a subject with cancer, and where the cell expressing CD20 at low density is a normal B cell in the subject with cancer. The cancer can be, for example, a CD20-positive leukemia, lymphoma, or myeloma. In certain embodiments, the subject is human.

The disclosure further provides a method for treating cancer in a subject, where the method includes administering an effective amount of the provided binding molecule, the provided composition, or the provided IgM or IgM-like antibody to a subject in need of treatment.

The disclosure further provides for the use of an effective amount of the provided binding molecule, the provided composition, or the provided IgM or IgM-like antibody in the preparation of a medicament for treating cancer.

The disclosure further provides the provided binding molecule, the provided composition, or the provided IgM or IgM-like antibody, for use in treating cancer.

### BRIEF DESCRIPTION OF THE DRAWINGS/FIGURES

FIGS. 1A-1D: Generation of anti-CD20 IgGs and IgMs with altered binding affinity. FIG. 1A. Non-reduced gel shows assembled IgGs. FIG. 1B. Reduced gel shows IgG heavy and light chains. For FIGS. 1A and 1B: Lane 1: RTX-IgG WT, Lane 2: RTX IgG N93A, Lane 3: RTX IgG N93D, Lane 4: RTX IgG N93E, Lane 5: RTX IgG N93K, Lane 6: RTX IgG N93R. FIG. 1C. Non-reduced gel shows assembled IgMs. FIG. 1D. Reduced gel shows IgM heavy and light chains. FIGS. 1C and 1D: Lane 1: RTX-IgM+J WT, Lane 2: RTX IgM+J N93A, Lane 3: RTX IgM+J N93D, Lane 4: RTX IgM+J N93E, Lane 5: RTX IgM+J N93K, Lane 6: RTX IgM+J N93R.

FIGS. 2A-2B: Binding activity of various altered-affinity mutants of rituximab to Ramos cells as IgG (FIG. 2A) and as IgM+J (FIG. 2B).

FIGS. 3A-3D: CDC activity of various altered-affinity mutants of rituximab on Ramos and CA46 B cell lines. FIG. 3A: CDC activity of IgG antibodies on Ramos cells;

FIG. 3B: CDC Activity of IgM antibodies on Ramos cells; FIG. 3C: CDC activity of IgG antibodies on CA46 cells; FIG. 3D: CDC activity of IgM antibodies on CA46 cells.

FIGS. 4A-4C: TDCC activity of two altered affinity mutants of rituximab on CA46 cells (FIG. 4A), normal B cells (FIG. 4B), and Ramos cells (FIG. 4C). Open circles: RTX-IGM V15J; open diamonds: RTX-IgM N93E V15J; open triangles: RTX-IgM N93D V15J.

### DETAILED DESCRIPTION

### Definitions

The terms “a” or “an” entity refer to one or more of that entity; for example, “a binding molecule,” represents one or more binding molecules. As such, terms such as “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions, or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the binding molecule, e.g., antibody binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, a gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, a polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, the term “binding molecule” refers in its broadest sense to a molecule that specifically binds to a binding target, e.g., an epitope or an antigenic determinant. As described further herein, a binding molecule can comprise one of more “antigen-binding domains” described herein. A non-limiting example of a binding molecule is an antibody or antibody-like molecule as described in detail herein that retains antigen-specific binding. In certain embodiments a “binding molecule” comprises an antibody or antibody-like molecule as described in detail herein.

As used herein, the terms “binding domain” or “antigen-binding domain” (can be used interchangeably) refer to a region of a binding molecule, e.g., an antibody or antibody-like molecule, that is necessary and sufficient to specifically bind to a binding target, e.g., an epitope. For example, an “Fv,” e.g., a heavy chain variable region and a light chain variable region of an antibody, either as two separate polypeptide subunits or as a single chain, is considered to be a “binding domain.” Other antigen-binding domains include, without limitation, the heavy chain variable region (VHH) of an antibody derived from a camelid species, a VNAR antigen receptor from sharks, or six immunoglobulin complementarity determining regions (CDRs) expressed in a heterologous scaffold, e.g., a fibronectin scaffold. A “binding molecule,” or “antibody” as described herein can include one, two, three, four, five, six, seven, eight, nine, ten, eleven, twelve, or even more “antigen-binding domains.” As used herein, a “binding unit-associated antigen-binding domain” refers to an antigen binding domain that is part of an antibody heavy chain and/or an antibody light chain. The term “J-chain-associated antigen-binding domain” refers to an antigen binding domain that is associated with a modified J-chain as described herein, for example, a ScFv fused to a wild type human J-chain, or functional fragment or variant thereof.

The terms “antibody” and “immunoglobulin” can be used interchangeably herein. An antibody (or a fragment, variant, or derivative thereof as disclosed herein) includes at least the variable region of a heavy chain (for camelid species) or at least the variable regions of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and a J-chain or functional fragment or variant thereof and/or a secretory component, or an IgM antibody that includes ten or twelve complete heavy chains and ten or twelve complete light chains and optionally includes a J-chain or functional fragment or variant thereof.

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2). It is the nature of this chain that determines the “isotype” of the antibody as IgG, IgM, IgA IgD, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins would be readily discernible to the skilled artisan in view of this disclosure and, accordingly, are within the scope of this disclosure.

Light chains are classified as either kappa or lambda (κ, λ). Each heavy chain class can associate with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, antigen-binding fragment thereof, or multimerizing fragment thereof, which corresponds to a standard “H2L2” immunoglobulin structure, e.g., two heavy chains or fragments thereof and two light chains or fragments thereof. In certain embodiments a binding unit can correspond to two heavy chains, e.g., in a camelid antibody. In certain embodiments, e.g., where the binding molecule is a bivalent IgG antibody or antigen-binding fragment thereof, the terms “binding molecule” and “binding unit” are equivalent. In other embodiments, e.g., where the binding molecule is multimeric, e.g., a dimeric IgA antibody or IgA-like antibody, a pentameric IgM antibody or IgM-like antibody, or a hexameric IgM antibody or IgM-like antibody, the binding molecule comprises two or more “binding units.” Two in the case of an IgA dimer, or five or six in the case of an IgM pentamer or hexamer, respectively. A binding unit need not include full-length antibody heavy and light chains, but will typically be bivalent, i.e., will include two “binding unit-associated antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments thereof. A binding molecule, e.g., an antibody or antibody-like molecule, comprising two or more, e.g., two, five, or six binding units, is referred to herein as “multimeric.” By “multimerizing fragment” is meant a portion of an IgM or IgA constant region that is sufficient to form a pentamer or hexamer in the case of IgM or a dimer in the case of IgA. IgM and IgA constant region fragments sufficient to multimerize are described elsewhere herein.

The term “J-chain” as used herein refers to the J-chain of native sequence IgM or IgA antibodies of any animal species, any functional fragment thereof, derivative thereof, and/or variant thereof, including the mature human J-chain, the amino acid sequence of which is presented as SEQ ID NO: 42. Various J-chain variants and modified J-chain derivatives are disclosed herein. As used herein, a “functional fragment” or a “functional variant” includes those fragments and variants that can associate with IgM heavy chain constant regions to form a pentameric IgM antibody (or alternatively can associate with IgA heavy chain constant regions to form a dimeric IgA antibody).

The term “modified J-chain” is used herein to refer to a derivative of a native sequence J-chain polypeptide comprising a heterologous moiety, e.g., a heterologous polypeptide, e.g., an extraneous antigen-binding domain introduced into the native J-chain sequence or a variant J-chain sequence. The introduction can be achieved by any means, including fusion of the heterologous polypeptide or other moiety or by attachment through a peptide or chemical linker. The term “modified human J-chain” encompasses, without limitation, a native sequence human J-chain comprising the amino acid sequence of SEQ ID NO: 42 or functional fragment thereof, or functional variant thereof, modified by the introduction of a heterologous moiety, e.g., a heterologous polypeptide, e.g., a J-chain-associated antigen-binding domain. In certain embodiments, the heterologous moiety does not interfere with efficient polymerization of IgM monomers into a pentamer and binding of pentameric IgM to a target. Exemplary modified J-chains can be found, e.g., in U.S. Pat. Nos. 9,951,134 and 10,618,978 and in U.S. Patent Application Publication No. US-2019-0185570, each of which is incorporated herein by reference in its entirety.

As used herein, the terms “IgM-derived binding molecule,” “IgM-like antibody,” “IgM-like binding unit,” or “IgM-like heavy chain constant region” refer to a variant antibody-derived binding molecule, antibody, binding unit, or heavy chain constant region that still retains the structural portions of an IgM heavy chain necessary to confer the ability to form multimers, i.e., hexamers, or in association with J-chain, form pentamers. An IgM-like antibody or IgM-derived binding molecule typically includes at least the Cμ4 and tailpiece (tp) domains of the IgM constant region but can include heavy chain constant region domains from other antibody isotypes, e.g., IgG, from the same species or from a different species. An IgM-like antibody or IgM-derived binding molecule can likewise be an antibody fragment in which one or more constant region domains are deleted, as long as the IgM-like antibody is capable of forming hexamers and/or pentamers. Thus, an IgM-like antibody or IgM-derived binding molecule can be, e.g., a hybrid IgM/IgG antibody or can be a “multimerizing fragment” of an IgM antibody.

As used herein, the terms “IgA-derived binding molecule,” “IgA-like antibody,” “IgA-like binding unit,” or “IgA-like heavy chain constant region” refer to a variant antibody-derived binding molecule, antibody, binding unit, or heavy chain constant region that still retains the structural portions of an IgA heavy chain necessary to confer the ability to form multimers, i.e., dimers, in association with J-chain. An IgA-like antibody or IgA-derived binding molecule typically includes at least the Cα3 and tailpiece (tp) domains of the IgA constant region but can include heavy chain constant region domains from other antibody isotypes, e.g., IgG, from the same species or from a different species. An IgA-like antibody or IgA-derived binding molecule can likewise be an antibody fragment in which one or more constant regions are deleted, as long as the IgA-like antibody is capable of forming dimers in association with a J-chain. Thus, an IgA-like antibody or IgA-derived binding molecule can be, e.g., a hybrid IgA/IgG antibody or can be a “multimerizing fragment” of an IgA antibody.

The terms “valency,” “bivalent,” “multivalent” and grammatical equivalents, refer to the number of antigen-binding domains in given binding molecule, e.g., antibody or antibody-like molecule, or in a given binding unit. As such, the terms “bivalent”, “tetravalent”, and “hexavalent” in reference to a given binding molecule, e.g., an IgM antibody, IgM-like antibody or multimerizing fragment thereof, denote the presence of two antigen-binding domains, four antigen-binding domains, and six antigen-binding domains, respectively. A typical IgM antibody or IgM-like antibody or IgM-derived binding molecule where each binding unit is bivalent, can have 10 or 12 valencies, or eleven valencies if a pentameric IgM comprises a modified J-chain that includes a single J-chain-associated antigen-binding domain. A bivalent or multivalent binding molecule, e.g., antibody or antibody-like molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody or antibody-like molecule. In certain embodiments, an epitope can include chemically active surface groupings of molecules such as amino acids, sugar side chains, phosphoryl, or sulfonyl, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., antibody or antibody-like molecule. A target can be, e.g., a polypeptide, a nucleic acid, a carbohydrate, a lipid, or other molecule. Moreover, a “target” can, for example, be a cell, an organ, or an organism that comprises an epitope that can be bound by a binding molecule, e.g., antibody or antibody-like molecule. As used herein, a “target antigen” is a target molecule, e.g., a polypeptide, a nucleic acid, a carbohydrate, a lipid, or other molecule that can be bound by a binding molecule, e.g., an antibody or antibody-like molecule as provided herein. In certain embodiments a target antigen can appear on the surface of a cell, e.g., a tumor cell. A “tumor-specific antigen” as used herein is a protein or other cell surface target antigen that is unique to tumor cells, at least at later stages of development of the organism. As used herein, a “tumor-associated antigen” is a protein or other cell surface target antigen that is not necessarily unique to tumor cells but is typically expressed much more abundantly and/or at higher density on tumor cells than on normal, healthy cells.

Both the light and heavy chains are divided into regions of structural and functional homology. The terms “constant” and “variable” are used functionally. The variable regions of both the light (VL) and heavy (VH) chains determine antigen recognition and specificity. Conversely, the constant domains of the light chain (CL) and the heavy chain (e.g., CH1, hinge, CH2, CH3, and/or CH4) confer biological properties such as molecule flexibility, secretion, transplacental mobility, Fc receptor binding, complement binding, and the like. By convention, the numbering of the constant region domains increases as they become more distal from the antigen-binding site or amino-terminus of the antibody heavy chain subunit. The N-terminal portion is a variable region (VH) and at the C-terminal portion is a constant region; for example, the CH3 and tail piece in the case of IgA or CH4 and tail piece in the case of IgM and CL domains comprise the carboxy-terminus of the heavy and light chain, respectively.

A “full length IgM antibody heavy chain” is a polypeptide that includes, in N-terminal to C-terminal direction, an antibody heavy chain variable region (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) and can further include a tailpiece.

A “full length IgA antibody heavy chain” is a polypeptide that includes, in N-terminal to C-terminal direction, an antibody heavy chain variable region (VH), an antibody constant heavy chain constant domain 1 (CA1 or Cα1), an IgA hinge region, an antibody heavy chain constant domain 2 (CA2 or Cα2), and an antibody heavy chain constant domain 3 (CA3 or Cα3) and can further include a tailpiece.

As indicated above, variable region(s) allows a binding molecule, e.g., antibody or antibody-like molecule, to selectively recognize and specifically bind epitopes on antigens. That is, the VL domain and VH domain, or subset of the complementarity determining regions (CDRs), of a binding molecule, e.g., an antibody or antibody-like molecule, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the R-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the target antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable regions can also be analyzed, e.g., using the IMGT information system (imgt_dot_cines_dot_fr/) (IMGT®/V-Quest) to identify variable region segments, including CDRs. (See, e.g., Brochet et al., Nucl. Acids Res., 36:W503-508, 2008).

Kabat et al. also defined a numbering system for variable region and constant region sequences that is applicable to any antibody. One of ordinary skill in the art can unambiguously assign this system of “Kabat numbering” to any variable region sequence, without reliance on any experimental data beyond the sequence itself. As used herein,

“Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 2 (allele IGHM*04)) and by the Kabat system is set out below. The underlined amino acid residues are not accounted for in the Kabat system (“X,” double underlined below, can be serine (S) (SEQ ID NO: 1) or glycine (G) (SEQ ID NO: 2)):

| Sequential (SEQ ID NO: 1 or SEQ ID NO: 2)/KABAT numbering key | |
|---|---|
| for IgM heavy chain | |
| 1/127 | GSASAPTLFP LVSCENSPSD TSSVAVGCLA QDFLPDSITF SWKYKNNSDI |
| 51/176 | SSTRGFPSVL RGGKYAATSQ VLLPSKDVMQ GTDEHVVCKV QHPNGNKEKN |
| 101/226 | VPLPVIAELP PKVSVFVPPR DGFFGNPRKS KLICQATGFS PRQIQVSWLR |
| 151/274 | EGKQVGSGVT TDQVQAEAKE SGPTTYKVTS TLTIKESDWL XQSMFTCRVD |
| 201/324 | HRGLTFQQNA SSMCVPDQDT AIRVFAIPPS FASIFLTKST KLTCLVTDLT |
| 251/374 | TYDSVTISWT RQNGEAVKTH TNISESHPNA TFSAVGEASI CEDDWNSGER |
| 301/424 | FTCTVTHTDL PSPLKQTISR PKGVALHRPD VYLLPPAREQ LNLRESATIT |
| 351/474 | CLVTGFSPAD VFVQWMQRGQ PLSPEKYVTS APMPEPQAPG RYFAHSILTV |
| 401/524 | SEEEWNTGET YTCVVAHEAL PNRVTERTVD KSTGKPTLYN VSLVMSDTAG |
| 451/574 | TCY |

Binding molecules, e.g., antibodies, antibody-like molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (ScFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody or antibody-like molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹ 5×10⁻⁴ sec⁻¹, 10⁻⁴ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule, e.g., an antibody can be said to competitively inhibit binding of the reference antibody or antigen binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. The affinity of an antigen-binding domain, e.g., a Fab fragment, for an antigen can be described or specified as a dissociation constant or K_{D}. K_{D} is the equilibrium dissociation constant, a ratio of koff/kon, between the antibody and its antigen. K_{D} and affinity are inversely related. The lower the K_{D} value the higher the affinity of the antibody. In certain embodiments a binding molecule, e.g., an antibody as provided herein has a dissociation constant or K_{D} no greater than 5×10⁻² M, 10⁻² M, 5×10⁻³M, 10⁻³M, 5×10⁻⁴M, 10⁻⁴M, 5×10⁻⁵M, 10⁻⁵ M, 5×10⁻⁶ M, 10⁻⁶M, 5×10⁻⁷ M, 10⁻⁷ M, 5×10⁻⁸M, 10⁻⁸ M, 5×10⁻⁹ M, 10⁻⁹ M, 5×10⁻¹⁰ M, 10⁻¹⁰ M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴ M, 5×10⁻¹⁵M, or 10⁻¹⁵M. In certain embodiments a binding molecule, e.g., an antibody or antigen binding fragment thereof as provided herein has enhanced selectivity for a cell that expresses a target antigen at higher density, e.g., a tumor-associated antigen expressed on a tumor cell. By “enhanced selectivity” is meant that the provided binding molecule, e.g., antibody binds to target cells that express a preselected target antigen at higher density than other cells that likewise express the antigen. For example, tumor cells or malignant cells often express a tumor-associated antigen that is also expressed on normal, healthy cells, but the tumor cells express the target antigen at much higher levels or higher density than on the normal cells. In order to obtain enhanced selectivity, a binding molecule, e.g., antibody as provided herein, can be modified to have more than two antigen-binding domains, e.g., at least three, at least four, at least five, at least six, at least seven, at least eight, at least nine, at least ten, at least eleven or twelve antigen-binding domains that have a lower affinity for a predetermined target antigen, i.e., a higher K_{D}, than the equivalent antigen-binding domains of a known monomeric bivalent reference antibody. For example, an IgA or IgM antibody with enhanced selectivity for a tumor cell versus a normal healthy cell can have more than two antigen-binding domains, e.g., at least three, at least four, at least five, at least six, at least seven, at least eight, at least nine, at least ten, at least eleven or twelve antigen-binding domains, comprising a K_{D} at least 5-fold, at least 10-fold, at least 50-fold, at least 100-fold, at least 500-fold, at least 1000-fold, at least 5000-fold or at least 10,000-fold or more greater than the K_{D} of antigen-binding domains of a known monomeric divalent antibody that binds to the predetermined target antigen.

As used herein, the term “avidity” refers to the overall stability of the complex between a population of antigen-binding domains and an antigen, or a complex of antigens. See, e.g., Harlow at pages 29-34. Avidity is related to both the affinity of the individual antigen-binding domains in the population with specific epitopes, and the valencies of immunoglobulins and the antigen. For example, a decavalent IgM antibody would bind to an antigen with higher avidity than a bivalent IgG antibody with antigen-binding domains having equivalent binding affinities. Avidity can also be affected by the antigen—for example, the interaction between a bivalent IgG antibody and an antigen with a highly repeating epitope structure, such as a polymer, would be one of high avidity. An interaction between a bivalent, tetravalent, or decavalent antibody with a receptor present at a high density on a cell surface would also be of high avidity. Where an antibody is binding to a target antigen present on the surface of a cell, e.g., a tumor-specific or tumor-associated antigen on a tumor cell, at least three levers can affect avidity: (a) the valency of the antibody, e.g., a bivalent IgG antibody will have a different avidity than a decavalent IgM antibody with equivalent antigen-binding domains; (b) an increase or decrease in the affinity of individual antigen-binding domains of the antibody, e.g., two, three, four, five, six, seven, eight, nine, ten, eleven, or twelve antigen-binding domain, and/or (c) the density of the target antigen of the surface of the cell, e.g., the difference between a tumor cell which expresses a tumor-associated antigen at high density versus a normal healthy cell that expresses the target antigen at lower density. This disclosure provides antibodies in which levers (a) and (b) can be manipulated to provide enhanced selectivity for diseased cells expressing a target antigen at high density versus normal cells that express the target antigen at lower density.

Binding molecules, e.g., antibodies or antibody-like molecules as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

“Antigen-binding antibody fragments,” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be, e.g., human, murine, donkey, rabbit, goat, guinea pig, camel, llama, horse, bear, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, an IgM or IgM-like antibody, an IgA or IgA-like antibody, or an IgM- or IgA-derived binding molecule as provided herein can include an antigen-binding fragment of an antibody, e.g., a ScFv fragment, so long as it is able to form a multimer, e.g., a dimer, hexamer, or pentamer.

As used herein, the term “heavy chain subunit” includes amino acid sequences derived from an immunoglobulin heavy chain. A heavy chain subunit can include at least one of: a VH domain, a CH1 domain, a hinge (e.g., upper, middle, and/or lower hinge region) domain, a CH2 domain, a CH3 domain, a CH4 domain, a tail-piece (tp), or a variant or fragment thereof. Further, a heavy chain subunit can lack certain constant region portions, e.g., all or part of a CH1 domain and/or a CH2 domain. These domains (e.g., the heavy chain subunit) can be modified such that they vary in amino acid sequence from the original heavy chain subunit. According to embodiments of the present disclosure, a multimeric binding molecule that includes an IgM or IgM-like heavy chain or an IgA or IgA-like heavy chain as provided herein includes sufficient portions of an IgM, IgM-like, IgA, or IgA-like heavy chain constant region(s) to allow the binding molecule to form a multimer, e.g., a dimer, hexamer, or pentamer, e.g., the heavy chain constant region includes a “multimerizing fragment” of an IgM, IgM-like, IgA, or IgA-like heavy chain constant region.

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies, antibody-like molecules, antigen-binding fragments, variants, or derivatives thereof, or multimerizing fragments thereof can be described or specified in terms of the epitope(s) or portion(s) of an antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein, the term “hinge region” includes the portion of a heavy chain molecule that joins the CH1 domain to the CH2 domain in IgG, IgA, and IgD heavy chains. This hinge region comprises approximately 25 amino acids and is flexible, thus allowing the two N-terminal antigen binding regions to move independently.

As used herein the term “disulfide bond” includes the covalent bond formed between two sulfur atoms. The amino acid cysteine comprises a thiol group that can form a disulfide bond or bridge with a second thiol group.

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody” or “bispecific antibody” refer to an antibody or antibody-like molecule that has antigen-binding domains for two or more different epitopes within a single antibody molecule. Other binding molecules in addition to the canonical antibody structure can be constructed with two binding specificities.

As used herein, the term “engineered antibody” refers to an antibody in which the variable region in either the heavy and light chain or both is altered by at least partial replacement of one or more amino acids in either the CDR or framework regions. In certain embodiments, a binding molecule, e.g., an antibody as provided herein can be engineered to have a reduced affinity for a predetermined target antigen than that of a known reference antibody, e.g., an approved therapeutic antibody or a therapeutic antibody in development. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments, not all of the CDRs are replaced with the complete CDRs from the donor variable region and yet the antigen binding capacity of the donor can still be transferred to the recipient variable regions. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides, nucleic acids, or glycans, or some combination of these techniques).

As used herein, the terms “linked,” “fused,” “fusion” or other grammatical equivalents can be used interchangeably. These terms refer to the joining together of two more elements, or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in atypical antibody, the variable region is “N-terminal” to the constant region, and the constant region is “C-terminal” to the variable region.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Terms such as “treating” or “treatment” or “to treat” or “alleviating” or “to alleviate” refer to therapeutic measures that cure, slow down, lessen symptoms of, and/or halt or slow the progression of an existing diagnosed disease, pathologic condition, or disorder. Terms such as “prevent,” “prevention,” “avoid,” “deterrence” and the like refer to prophylactic or preventative measures that prevent the development of an undiagnosed targeted disease, pathologic condition, or disorder. Thus, “a subject in need of treatment” can include subjects already with the disorder; those prone to have the disorder; and those in whom the disorder is to be prevented.

As used herein the terms “serum half-life” or “plasma half-life” refer to the time it takes (e.g., in minutes, hours, or days) following administration for the serum or plasma concentration of a protein or a drug, e.g., a binding molecule such as an antibody or antibody-like molecule as described herein, to be reduced by 50%. Two half-lives can be described: the alpha half-life or a half-life, or t_{1/2}α, which is the rate of decline in plasma concentrations due to the process of drug redistribution from the central compartment, e.g., the blood in the case of intravenous delivery, to a peripheral compartment (e.g., a tissue or organ), and the beta half-life or 3 half-life, or t_{1/2}β, which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve is measured from time 0 (t₀) to infinity (∞) and is dependent on the rate of elimination of the drug from the body and the dose administered.

As used herein, the term “mean residence time” or “MRT” refers to the average length of time the drug remains in the body.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” refers to a subset of subjects, from amongst all prospective subjects, which would benefit from administration of a given therapeutic agent, e.g., a binding molecule such as an antibody or antibody-like molecule, comprising one or more antigen-binding domains. Such binding molecules, e.g., antibodies or antibody-like molecules, can be used, e.g., for a diagnostic procedure and/or for treatment or prevention of a disease.

IgM Antibodies, IgM-Like Antibodies, and IgM-Derived Binding Molecules

IgM is the first immunoglobulin produced by B cells in response to stimulation by antigen and is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (p) constant region of IgM additionally contains a fourth constant domain (CH4) and includes a C-terminal “tailpiece” (tp). While several human alleles exist, the human IgM constant region typically comprises the amino acid sequence SEQ ID NO: 1 (IMGT allele IGHM*03, identical to, e.g., GenBank Accession No. pir∥S37768) or SEQ ID NO: 2 (IMGT allele IGHM*04, identical to, e.g., GenBank Accession No. sp∥P01871.4). The human Cμ1 region ranges from about amino acid 5 to about amino acid 102 of SEQ ID NO: 1 or SEQ ID NO: 2; the human Cμ2 region ranges from about amino acid 114 to about amino acid 205 of SEQ ID NO: 1 or SEQ ID NO: 2, the human Cμ3 region ranges from about amino acid 224 to about amino acid 319 of SEQ ID NO: 1 or SEQ ID NO: 2, the Cp 4 region ranges from about amino acid 329 to about amino acid 430 of SEQ ID NO: 1 or SEQ ID NO: 2, and the tailpiece ranges from about amino acid 431 to about amino acid 453 of SEQ ID NO: 1 or SEQ ID NO: 2.

Other forms of the human IgM constant region with minor sequence variations exist, including, without limitation, GenBank Accession Nos. CAB37838.1 and pir∥MHHU. The amino acid substitutions, insertions, and/or deletions at positions corresponding to SEQ ID NO: 1 or SEQ ID NO: 2 described and claimed elsewhere in this disclosure can likewise be incorporated into alternate human IgM sequences, as well as into IgM constant region amino acid sequences of other species.

Each IgM heavy chain constant region can be associated with an antigen-binding domain, e.g., a ScFv or VHH, or a subunit of an antigen-binding domain, e.g., a VH region.

Five IgM binding units can form a complex with an additional small polypeptide chain (the J-chain or a functional fragment, variant, or derivative thereof) to form a pentameric IgM antibody or IgM-like antibody with ten binding unit-associated antigen-binding domains. In the precursor form of the human J-chain, the signal peptide (underlined) extends from amino acid 1 to about amino acid 22 of SEQ ID NO: 6, and the mature human J-chain extends from about amino acid 23 to amino acid 159 of SEQ ID NO: 6. The mature human J-chain has the amino acid sequence SEQ ID NO: 42.

Exemplary variant and modified J-chains are provided elsewhere herein. Without the J-chain, an IgM antibody or IgM-like antibody typically assembles into a hexamer, comprising six binding units and up to twelve binding unit-associated antigen-binding domains. With a J-chain, an IgM antibody or IgM-like antibody typically assembles into a pentamer, comprising five binding units and up to ten binding unit-associated antigen-binding domains, or more, if the J-chain is a modified J-chain comprising one or more heterologous polypeptides that can be, e.g., J-chain-associated antigen-binding domain(s). The assembly of five or six IgM binding units into a pentameric or hexameric IgM antibody or IgM-like antibody is thought to involve interactions between the Cμ4 and tailpiece domains of the five or six binding units. See, e.g., Braathen, R., et al., J Biol. Chem. 277:42755-42762 (2002). Accordingly, the constant regions of a pentameric or hexameric IgM antibody or antibody-like molecule provided in this disclosure typically includes at least the Cμ4 and/or tailpiece domains (also referred to herein collectively as Cμ4-tp). A “multimerizing fragment” of an IgM heavy chain constant region thus includes at least the Cμ4-tp domain. An IgM heavy chain constant region can additionally include a Cμ3 domain or a fragment thereof, a Cμ2 domain or a fragment thereof, a Cμ1 domain or a fragment thereof. In certain embodiments, a binding molecule, e.g., an IgM antibody or IgM-like antibody as provided herein can include a complete IgM heavy (p) chain constant domain, e.g., SEQ ID NO: 1 or SEQ ID NO: 2, or a variant, derivative, or analog thereof, e.g., as provided herein.

In certain embodiments, the disclosure provides a pentameric IgM or IgM-like antibody comprising five bivalent binding units, where each binding unit includes two IgM heavy chain constant regions or multimerizing fragments or variants thereof, each associated with an antigen-binding domain or a subunit of an antigen-binding domain. In certain embodiments, the two IgM heavy chain constant regions are human heavy chain constant regions.

Where the IgM or IgM-like antibody provided herein is pentameric, the IgM or IgM-like antibody typically further includes a J-chain, or functional fragment or variant thereof. In certain embodiments, the J-chain can be a modified J-chain comprising, e.g., a J-chain-associated antigen binding domain that specifically binds to a target, e.g., an immune effector cell, e.g., a CD8+ cytotoxic T cell or an NK cell. In certain embodiments the modified J-chain can further comprise one or more heterologous moieties attached thereto, e.g., an immune stimulatory agent. In certain embodiments the J-chain can be mutated to affect, e.g., enhance, the serum half-life of the IgM or IgM-like antibody provided herein, as discussed elsewhere herein. In certain embodiments the J-chain can be mutated to affect glycosylation, as discussed elsewhere in this disclosure.

In some embodiments, the multimeric binding molecules are hexameric and comprise six bivalent binding units or variants or fragments thereof. In some embodiments, the multimeric binding molecules are hexameric and comprise six bivalent binding units or variants or fragments thereof, and where each binding unit comprises two IgM heavy chain constant regions or multimerizing fragments or variants thereof.

An IgM heavy chain constant region can include one or more of a Cμ1 domain or fragment or variant thereof, a Cμ2 domain or fragment or variant thereof, a Cμ3 domain or fragment or variant thereof, a Cμ4 domain or fragment or variant thereof, and/or a tail piece (tp) or fragment or variant thereof, provided that the constant region can serve a desired function in the IgM or IgM-like antibody, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a tp or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In some embodiments, the binding units of the IgM or IgM-like antibody comprise two light chains. In some embodiments, the binding units of the IgM or IgM-like antibody comprise two fragments of light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

IgM Antibodies, IgM-Like Antibodies, and IgM-Derived Binding Molecules with Enhanced Serum Half-Life

Certain IgM-derived multimeric bispecific binding molecules provided herein can be modified to have enhanced serum half-life. Exemplary IgM heavy chain constant region mutations that can enhance serum half-life of an IgM-derived binding molecule are disclosed in PCT Publication No. WO 2019/169314, which is incorporated by reference herein in its entirety. For example, a variant IgM heavy chain constant region of an IgM-derived binding molecule as provided herein can include an amino acid substitution at an amino acid position corresponding to amino acid S401, E402, E403, R344, and/or E345 of a wild-type human IgM constant region (e.g., SEQ ID NO: 1 or SEQ ID NO: 2). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 1 or SEQ ID NO: 2 can be substituted with any amino acid, e.g., alanine.

IgM Antibodies, IgM-Like Antibodies, and IgM-Derived Binding Molecules with Reduced CDC Activity

Certain IgM-derived multimeric binding molecules as provided herein can be engineered to exhibit reduced complement-dependent cytotoxicity (CDC) activity to cells in the presence of complement, relative to a reference IgM antibody or IgM-like antibody with a corresponding reference human IgM constant region identical, except for the mutations conferring reduced CDC activity. These CDC mutations can be combined with any of the mutations to confer increased serum half-life as provided herein. By “corresponding reference human IgM constant region” is meant a human IgM constant region or portion thereof, e.g., a Cμ3 domain, that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in PCT Publication No. WO/2018/187702, which is incorporated herein by reference in its entirety. Assays for measuring CDC are well known to those of ordinary skill in the art, and exemplary assays are described e.g., in PCT Publication No. WO/2018/187702.

In certain embodiments, a variant human IgM constant region conferring reduced CDC activity includes an amino acid substitution corresponding to the wild-type human IgM constant region at position L310, P311, P313, and/or K315 of SEQ ID NO: 1 (human IgM constant region allele IGHM*03) or SEQ ID NO: 2 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 1 or SEQ ID NO: 2. In other embodiments the variant IgM constant region as provided herein contains an amino acid substitution corresponding to the wild-type human IgM constant region at position P313 of SEQ ID NO: 1 or SEQ ID NO: 2. In other embodiments the variant IgM constant region as provided herein contains a combination of substitutions corresponding to the wild-type human IgM constant region at positions P311 of SEQ ID NO: 1 and/or SEQ ID NO: 2 and P313 of SEQ ID NO: 1 or SEQ ID NO: 2. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 1 or SEQ ID NO: 2 with aspartic acid. In certain embodiments, a variant human IgM constant region conferring reduced CDC activity includes an amino acid substitution corresponding to the wild-type human IgM constant region at position L310 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position L310 of SEQ ID NO: 1 or SEQ ID NO: 2 with aspartic acid.

Human and certain non-human primate IgM constant regions typically include five (5) naturally-occurring asparagine (N)-linked glycosylation motifs or sites. As used herein “an N-linked glycosylation motif” comprises or consists of the amino acid sequence N-X₁-S/T, where N is asparagine, X₁ is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 1 or SEQ ID NO: 2 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N-X₁-S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiment, the IgM constant region comprises one or more substitutions relative to a wild-type human IgM constant region at positions 46, 209, 272, or 440 of SEQ ID NO: 1 (human IgM constant region allele IGHM*03) or SEQ ID NO: 2 (human IgM constant region allele IGHM*04). See, e.g., U.S. Provisional Application No. 62/891,263, which is incorporated herein by reference in its entirety.

IgA Antibodies, IgA-Like Antibodies, and IgA-Derived Binding Molecules

IgA plays a critical role in mucosal immunity and comprises about 15% of total immunoglobulin produced. IgA can be monomeric or multimeric, forming primarily dimeric molecules, but can also assemble as trimers, tetramers, and/or pentamers. See, e.g., de Sousa-Pereira, P., and J. M. Woof, Antibodies 8:57 (2019).

In some embodiments, the multimeric binding molecules are dimeric and comprise two bivalent binding units or variants or fragments thereof. In some embodiments, the multimeric binding molecules are dimeric, comprise two bivalent binding units or variants or fragments thereof, and further comprise a J-chain or functional fragment or variant thereof as described herein. In some embodiments, the multimeric binding molecules are dimeric, comprise two bivalent binding units or variants or fragments thereof, and further comprise a J-chain or functional fragment or variant thereof as described herein, where each binding unit comprises two IgA heavy chain constant regions or multimerizing fragments or variants thereof.

In some embodiments, the multimeric binding molecules are tetrameric and comprise four bivalent binding units or variants or fragments thereof. In some embodiments, the multimeric binding molecules are tetrameric, comprise four bivalent binding units or variants or fragments thereof, and further comprise a J-chain or functional fragment or variant thereof as described herein. In some embodiments, the multimeric binding molecules are tetrameric, comprise four bivalent binding units or variants or fragments thereof, and further comprise a J-chain or functional fragment or variant thereof as described herein, where each binding unit comprises two IgA heavy chain constant regions or multimerizing fragments or variants thereof.

In certain embodiments, the multimeric binding molecule provided by this disclosure is a dimeric binding molecule that includes IgA heavy chain constant regions, or multimerizing fragments thereof, each associated with a binding unit-associated antigen-binding domain for a total of four binding unit-associated antigen-binding domains. As provided herein, an IgA antibody, IgA-derived binding molecule, or IgA-like antibody includes two binding units and a J-chain, e.g., a modified J-chain as described elsewhere herein. Each binding unit as provided comprises two IgA heavy chain constant regions or multimerizing fragments or variants thereof. In certain embodiments, at least three or all four binding unit-associated antigen-binding domains of the multimeric binding molecule bind to the same target antigen. In certain embodiments, at least three or all four binding unit-associated antigen-binding domains of the multimeric binding molecule are identical.

A bivalent IgA-derived binding unit includes two IgA heavy chain constant regions, and a dimeric IgA-derived binding molecule includes two binding units. IgA contains the following heavy chain constant domains, Cα1 (or alternatively CA1 or CH1), a hinge region, Cα2 (or alternatively CA2 or CH2), and Cα3 (or alternatively CA3 or CH3), and a C-terminal “tailpiece.” Human IgA has two subtypes, IgA1 and IgA2. The human IgA1 constant region typically includes the amino acid sequence SEQ ID NO: 3 The human Cα1 domain extends from about amino acid 6 to about amino acid 98 of SEQ ID NO: 3; the human IgA1 hinge region extends from about amino acid 102 to about amino acid 124 of SEQ ID NO: 3, the human Cα2 domain extends from about amino acid 125 to about amino acid 219 of SEQ ID NO: 3, the human Cα3 domain extends from about amino acid 228 to about amino acid 330 of SEQ ID NO: 3, and the tailpiece extends from about amino acid 331 to about amino acid 352 of SEQ ID NO: 3. The human IgA2 constant region typically includes the amino acid sequence SEQ ID NO: 4. The human Cα1 domain extends from about amino acid 6 to about amino acid 98 of SEQ ID NO: 4; the human IgA2 hinge region extends from about amino acid 102 to about amino acid 111 of SEQ ID NO: 4, the human Cα2 domain extends from about amino acid 113 to about amino acid 206 of SEQ ID NO: 4, the human Cα3 domain extends from about amino acid 215 to about amino acid 317 of SEQ ID NO: 4, and the tailpiece extends from about amino acid 318 to about amino acid 340 of SEQ ID NO: 4.

Two IgA binding units can form a complex with two additional polypeptide chains, the J-chain (e.g., SEQ ID NO: 42) and the secretory component (precursor, SEQ ID NO: 5, mature, amino acids 19 to 603 of SEQ ID NO: 5) to form a bivalent secretory IgA (sIgA)-derived binding molecule as provided herein. The assembly of two IgA binding units into a dimeric IgA-derived binding molecule is thought to involve the Cα3 and tailpiece domains. See, e.g., Braathen, R., et al., J. Biol. Chem. 277:42755-42762 (2002). Accordingly, a multimerizing dimeric IgA-derived binding molecule provided in this disclosure typically includes IgA constant regions that include at least the Cα3 and tailpiece domains. Four IgA binding units can likewise form a tetramer complex with a J-chain. A sIgA antibody can also form as a higher order multimer, e.g., a tetramer.

An IgA heavy chain constant region can additionally include a Cα2 domain or a fragment thereof, an IgA hinge region or fragment thereof, a Cα1 domain or a fragment thereof, and/or other IgA (or other immunoglobulin, e.g., IgG) heavy chain domains, including, e.g., an IgG hinge region. In certain embodiments, a binding molecule as provided herein can include a complete IgA heavy (a) chain constant domain (e.g., SEQ ID NO: 3 or SEQ ID NO: 4), or a variant, derivative, or analog thereof. In some embodiments, the IgA heavy chain constant regions or multimerizing fragments thereof are human IgA constant regions.

In certain embodiments each binding unit of a multimeric binding molecule as provided herein includes two IgA or IgA-like heavy chain constant regions or multimerizing fragments or variants thereof, each including at least an IgA Cα3 domain and an IgA tailpiece domain. In certain embodiments the IgA or IgA-like heavy chain constant regions can each further include an IgA Cα2 domain situated N-terminal to the IgA Cα3 and IgA tailpiece domains. For example, the IgA heavy chain constant regions can include amino acids 125 to 353 of SEQ ID NO: 3 or amino acids 113 to 340 of SEQ ID NO: 4. In certain embodiments the IgA or IgA-like heavy chain constant regions can each further include an IgA or IgG hinge region situated N-terminal to the IgA Cα2 domains. For example, the IgA heavy chain constant regions can include amino acids 102 to 353 of SEQ ID NO: 3 or amino acids 102 to 340 of SEQ ID NO: 4. In certain embodiments the IgA or IgA-like heavy chain constant regions can each further include an IgA Cα1 domain situated N-terminal to the IgA hinge region.

In some embodiments, each binding unit of an IgA antibody, IgA-like antibody, or other IgA-derived binding molecule comprises two light chains. In some embodiments, each binding unit of an IgA antibody, IgA-like antibody, or other IgA-derived binding molecule comprises two fragments light chains. In some embodiments, the light chains are kappa light chains. In some embodiments, the light chains are lambda light chains. In some embodiments the light chains are chimeric kappa-lambda light chains. In some embodiments, each binding unit comprises two immunoglobulin light chains each comprising a VL situated amino terminal to an immunoglobulin light chain constant region.

Modified and/or Variant J-chains

In certain embodiments, the multimeric binding molecule provided herein comprises a J-chain or functional fragment or variant thereof. In certain embodiments, the multimeric binding molecule provided herein is pentameric and comprises a J-chain or functional fragment or variant thereof. In certain embodiments, the multimeric binding molecule provided herein is a dimeric IgA molecule or a pentameric IgM molecule and comprises a J-chain or functional fragment or variant thereof. In some embodiments, the multimeric binding molecule can comprise a naturally occurring J-chain sequence, such as a mature human J-chain sequence (e.g., SEQ ID NO: 42). In some embodiments, the multimeric binding molecule can comprise a functional fragment of a naturally occurring or variant J-chain.

In certain embodiments, the J-chain of a pentameric an IgM or IgM-like antibody or a dimeric IgA or IgA-like antibody as provided herein can be modified, e.g., by introduction of a heterologous moiety, or two or more heterologous moieties, e.g., polypeptides, without interfering with the ability of the IgM or IgM-like antibody or IgA or IgA-like antibody to assemble and bind to its binding target(s). See U.S. Pat. Nos. 9,951,134 and 10,618,978, and U.S. Patent Application Publication No. US-2019-0185570, each of which is incorporated herein by reference in its entirety. Accordingly, IgM or IgM-like antibodies or IgA or IgA-like antibodies as provided herein, including bispecific or multispecific IgM or IgM-like antibodies or IgA or IgA-like antibodies, can include a modified J-chain or functional fragment or variant thereof that further includes a heterologous moiety, e.g., a heterologous polypeptide, introduced into the J-chain or fragment or variant thereof. In certain embodiments the heterologous moiety can be, without limitation, a peptide or polypeptide fused in frame, or a peptide, polypeptide, or other chemical or biological moiety chemically conjugated to the J-chain or fragment or variant thereof. In certain embodiments, a heterologous polypeptide is fused to the J-chain or functional fragment or variant thereof via a linker, e.g., a peptide linker consisting of least 5 amino acids, but typically no more than 50 amino acids, e.g., 5, 10, 15. 20. 25. 30, 35, 40, 45, or 50 amino acids. In certain embodiments, the peptide linker consists of GGGGS (SEQ ID NO: 57), GGGGSGGGGS (SEQ ID NO: 58), GGGGSGGGGSGGGGS (SEQ ID NO: 59), GGGGSGGGGSGGGGSGGGGS (SEQ ID NO: 60), or GGGGSGGGGSGGGGSGGGGSGGGGS (SEQ ID NO: 61). In certain embodiments the heterologous moiety can be a chemical or biological moiety conjugated to the J-chain. Heterologous moieties to be attached to a J-chain can include, without limitation, a J-chain-associated antigen binding domain, e.g., an antibody or antigen-binding fragment or subunit, e.g., a single chain Fv (ScFv) molecule, a stabilizing peptide that can increase the half-life of the IgM, IgM-like, IgA, or IgA-like antibody, or a chemical moiety such as a polymer or a cytotoxin. In some embodiments, heterologous moiety comprises a stabilizing peptide that can increase the half-life of the binding molecule, e.g., human serum albumin (HSA) or an HSA binding molecule.

In some embodiments, a modified J-chain includes a J-chain-associated antigen-binding domain, e.g., a polypeptide capable of specifically binding to a target antigen. In certain embodiments, a J-chain-associated antigen-binding domain can be an antibody or an antigen-binding fragment thereof, as described elsewhere herein. In certain embodiments the J-chain-associated antigen-binding domain can be a single chain Fv (scFv) antigen-binding domain or a single-chain antigen-binding domain derived, e.g., from a camelid or condricthoid antibody. The J-chain-associated antigen-binding domain can be introduced into the J-chain at any location that allows the binding of the J-chain-associated antigen-binding domain to its binding target without interfering with J-chain function or the function of an associated IgM, IgM-like, IgA, or IgA-like antibody. Insertion locations include but are not limited to at or near the C-terminus, at or near the N-terminus or at an internal location that, based on the three-dimensional structure of the J-chain, is accessible. In certain embodiments, the J-chain-associated antigen-binding domain can be introduced into the mature human J-chain of SEQ ID NO: 42 or a variant thereof between cysteine residues corresponding to amino acids 92 and 101 of SEQ ID NO: 42. In a further embodiment, the J-chain-associated antigen-binding domain can be introduced into the human J-chain of SEQ ID NO: 42 or a variant thereof at or near a glycosylation site. In a further embodiment, the J-chain-associated antigen-binding domain can be introduced into the human J-chain of SEQ ID NO: 42 or a variant thereof within 10 amino acid residues from the C-terminus, and/or within 10 amino acids from the N-terminus. In a further embodiment, the J-chain-associated antigen-binding domain can be introduced into the human J-chain of SEQ ID NO: 42 or a variant thereof at the C-terminus, and/or at the N-terminus.

In certain embodiments, the J-chain of an IgM antibody, IgM-like antibody, IgA antibody, IgA-like antibody, or IgM- or IgA-derived binding molecule as provided herein is a variant J-chain that comprises one or more amino acid substitutions that can alter, e.g., increase, the serum half-life of an IgM antibody, IgM-like antibody, IgA antibody, IgA-like antibody, or IgM- or IgA-derived binding molecule provided herein that includes the variant J-chain. For example certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain.

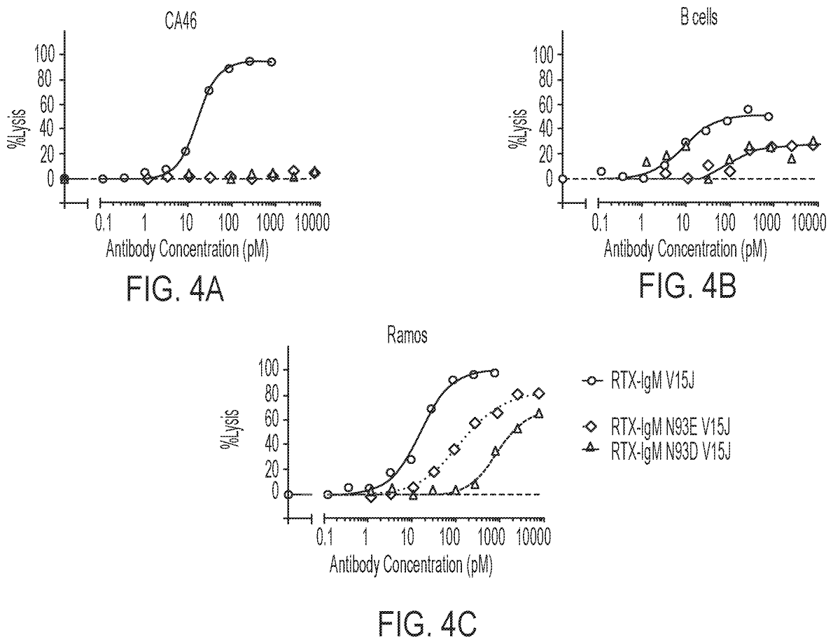

In some embodiments, the multimeric binding molecule can comprise a variant J-chain sequence, such as a variant sequence described herein with reduced glycosylation or reduced binding to one or more polymeric Ig receptors (e.g., pIgR, Fc alpha-mu receptor (FcαμR), or Fc mu receptor (FcμR)). See, e.g., PCT Publication No. WO 2019/169314, which is incorporated herein by reference in its entirety. In certain embodiments, the variant J-chain can comprise an amino acid substitution at the amino acid position corresponding to amino acid Y102 of the mature wild-type human J-chain (SEQ ID NO: 42). By “an amino acid corresponding to amino acid Y102 of the mature wild-type human J-chain” is meant the amino acid in the sequence of the J-chain of any species which is homologous to Y102 in the human J-chain. See PCT Publication No. WO/2019/169314, which is incorporated herein by reference in its entirety. The position corresponding to Y102 in SEQ ID NO: 42 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 42 can inhibit the binding of certain immunoglobulin receptors, e.g., the human or murine Fcαμ receptor, the murine Fcμ receptor, and/or the human or murine polymeric Ig receptor (pIg receptor) to an IgM pentamer comprising the variant J-chain. IgM antibodies, IgM-like antibodies, and IgM-derived binding molecules comprising a substitution at the amino acid corresponding to Y102 of SEQ ID NO: 42 have an improved serum half-life when administered to an animal than a corresponding antibody, antibody-like molecule or binding molecule that is identical except for the substitution, and which is administered to the same species in the same manner. In certain embodiments, the amino acid corresponding to Y102 of SEQ ID NO: 42 can be substituted with any amino acid. In certain embodiments, the amino acid corresponding to Y102 of SEQ ID NO: 42 can be substituted with alanine (A), serine (S) or arginine (R). In certain embodiments, the amino acid corresponding to Y102 of SEQ ID NO: 42 can be substituted with alanine. In a particular embodiment the J-chain or functional fragment or variant thereof is a variant human J-chain and comprises the amino acid sequence SEQ ID NO: 43, a J chain referred to herein as “J*.” See PCT Publication No. WO 2019/169314.

Wild-type J-chains typically include one N-linked glycosylation site. In certain embodiments, a variant J-chain or functional fragment thereof of a multimeric binding molecule as provided herein includes a mutation within the asparagine(N)-linked glycosylation motif N-X₁-S/T, e.g., starting at the amino acid position corresponding to amino acid 49 (motif N6) of the mature human J-chain (SEQ ID NO: 42) or J* (SEQ ID NO: 43), where N is asparagine, X₁ is any amino acid except proline, and S/T is serine or threonine, and where the mutation prevents glycosylation at that motif. As demonstrated in PCT Publication No. WO 2019/169314, mutations preventing glycosylation at this site can result in the multimeric binding molecule as provided herein, exhibiting an increased serum half-life upon administration to a subject animal relative to a reference multimeric binding molecule that is identical except for the mutation or mutations preventing glycosylation in the variant J-chain, and is administered in the same way to the same animal species.

For example, in certain embodiments the variant J-chain or functional fragment thereof of a binding molecule comprising a J-chain as provided herein can include an amino acid substitution at the amino acid position corresponding to amino acid N49 or amino acid S51 of SEQ ID NO: 42 or SEQ ID NO: 43, provided that the amino acid corresponding to S51 is not substituted with threonine (T), or where the variant J-chain comprises amino acid substitutions at the amino acid positions corresponding to both amino acids N49 and S51 of SEQ ID NO: 42 or SEQ ID NO: 43. In certain embodiments, the position corresponding to N49 of SEQ ID NO: 42 or SEQ ID NO: 43 is substituted with any amino acid, e.g., alanine (A), glycine (G), threonine (T), serine (S) or aspartic acid (D). In a particular embodiment, the position corresponding to N49 of SEQ ID NO: 42 or SEQ ID NO: 43 can be substituted with alanine (A). In another particular embodiment, the position corresponding to N49 of SEQ ID NO: 42 or SEQ ID NO: 43 can be substituted with aspartic acid (D). In some embodiments, the position corresponding to S51 of SEQ ID NO: 42 or SEQ ID NO: 43 is substituted with alanine (A) or glycine (G). In some embodiments, the position corresponding to S51 of SEQ ID NO: 42 or SEQ ID NO: 43 is substituted with alanine (A).

In certain embodiments, the J-chain-associated antigen-binding domain of the provided binding molecule includes an antibody or fragment thereof. In certain embodiments the antibody fragment is a Fab fragment, a Fab′ fragment, a F(ab′)2 fragment, a Fd fragment, a Fv fragment, a single-chain Fv (scFv) fragment, a disulfide-linked Fv (sdFv) fragment, or any combination thereof. In certain embodiments the antibody fragment is a single chain Fv (scFv) fragment. The scFv can be fused or chemically conjugated to the J-chain or fragment or variant, e.g., J*. In certain embodiments, the scFv fragment is fused to the J-chain via a peptide linker e.g., SEQ ID NO: 57-61. The scFv fragment can be fused to J-chain or fragment or variant thereof in any way so long as the ability of the modified J-chain to assemble with IgM, IgM-like, IgA, or IgA-like binding units to form a dimer or a pentamer, is not affected. For example the scFv fragment can be fused to the N-terminus of the J-chain or fragment or variant thereof, the C-terminus of the J-chain or fragment or variant thereof, or to both the N-terminus and C-terminus of the J-chain or fragment or variant thereof.

In certain embodiments the scFv of a modified J-chain binds to a target on an immune effector cell. The immune effector cell bound by the J-chain-associated antigen binding domain can be any immune effector cell that confers a beneficial effect when associated with binding unit-associated antigen-binding domains that target, e.g., a tumor-associated or tumor-specific target, for example mediating cell-based killing of tumor cells. In certain embodiments the immune effector cell can be, without limitation, a T cell, e.g., a CD4+ T cell, a CD8+ T cell, an NKT cell, or a γδ T cell, a B cell, a plasma cell, a macrophage, a dendritic cell, or a natural killer (NK) cell. In certain embodiments the immune effector cell is a T cell, e.g., a CD4+ or CD8+ T cell. In certain embodiments the immune effector cell is a CD8+ cytotoxic T cell. In certain embodiments the immune effector cell is an NK cell.

Where the immune effector cell is a T cell, for example a CD8+ T cell, the J-chain-associated scFv fragment can specifically bind to the T cell surface antigen CD3, e.g., CD3R. In certain embodiments the anti-CD3a scFv fragment comprises a heavy chain variable region (VH) and a light chain variable region (VL), wherein the VH comprises the VH complementarity-determining regions VHCDR1, VHCDR2, and VHCDR3 comprising the amino acid sequences SEQ ID NO: 49, SEQ ID NO: 50, and SEQ ID NO: 51, respectively, or SEQ ID NO: 49, SEQ ID NO: 50, and SEQ ID NO: 51 with one, two, or three amino acid substitutions in one or more of the VHCDRs, and wherein the VL comprises the VL complementarity-determining regions VLCDR1, VLCDR2, and VLCDR3 comprising the amino acid sequences SEQ ID NO: 53, SEQ ID NO: 54, and SEQ ID NO: 55, respectively, or SEQ ID NO: 53, SEQ ID NO: 54, and SEQ ID NO: 55 with one, two, or three amino acid substitutions in one or more of the VLCDRs. In certain embodiments, the ScFv fragment comprises the VH amino acid sequence SEQ ID NO: 48 and the VL amino acid sequence SEQ ID NO: 52. In other embodiments, the anti-CD3ε scFv fragment comprises a heavy chain variable region (VH) and a light chain variable region (VL), wherein the VH and VL comprise the amino acid sequences SEQ ID NO: 44 and SEQ ID NO: 45, respectively. In particular embodiments, the modified J chain comprises an amino acid sequence comprising amino acids 20 to 412 of SEQ ID NO: 46, amino acids 20 to 412 of SEQ ID NO: 47, or amino acids 20 to 420 of SEQ ID NO: 56.

In certain other embodiments, the immune effector cell is an NK cell, and the scFv fragment can specifically bind to CD16 or CD56.

A modified J-chain of a multimeric binding molecule as provided herein can be further modified to include additional heterologous moieties attached to the J-chain. Exemplary moieties are described, e.g., in U.S. Pat. Nos. 9,951,134 and 10,618,978, and in U.S. Patent Application Publication Nos. US 2019-0185570, and in PCT Application No. PCT/US2020/046379, all of which are incorporated herein by reference in their entireties. In certain embodiments, the modified J-chain of a multimeric binding molecule as provided herein can further include an immune stimulatory agent (“ISA”) fused or chemically conjugated to the J-chain or fragment or variant thereof. For example, the ISA can include a cytokine or receptor-binding fragment or variant thereof. In a particular embodiment, a J-chain-associated ISA can include (a) an interleukin-15 (IL-15) protein or receptor-binding fragment or variant thereof (“I”), and (b) an interleukin-15 receptor-α (IL-15Rα) fragment comprising the sushi domain or a variant thereof capable of associating with I (“R”), wherein the J-chain or fragment or variant thereof and at least one of I and R, or both I and R, are associated as a fusion protein, and wherein I and R can associate to function as the ISA. In certain embodiments, the ISA can be fused to the J-chain via a peptide linker.

Multivalent Antibodies with Enhanced Selectivity for Cells with High Target Density

This disclosure provides a multimeric binding molecule, e.g., an IgM, IgM-like, IgA, or IgA-like antibody with enhanced selectivity for binding to diseased cells, e.g., cancer cells or tumor cells, relative to normal healthy cells. The provided binding molecules can comprise two bivalent binding units (e.g., for a dimeric sIgA antibody), or five or six bivalent binding units (e.g., for a pentameric or hexameric IgM antibody). Each binding unit of the provided binding molecules typically includes two antibody heavy chains, each including an IgA, IgA-like, IgM, or IgM-like heavy chain constant region or multimerizing fragment or variant thereof, where the fragment(s) include at least the CH3 and tp domains of an IgA heavy chain constant region or the CH4 and tp domains of an IgM heavy chain constant region. Of course the heavy chains can include additional IgA or IgA-like heavy chain constant region domains (CH1, hinge, CH2) or IgM or IgM-like heavy chain constant region domains (CH1, CH2, CH3) or fragments thereof, and can also be hybrid heavy chain constant regions including, e.g., one or more IgG constant region domains (e.g., CH1, hinge, CH2, or CH3), or constant region domains of another antibody isotype or constant regions domains from another species. Each heavy chain constant region of a provided binding molecule can be associated with a binding unit-associated antigen-binding domain or subunit thereof, e.g., a heavy chain variable region (VH) that can associate with a light chain variable region (VL), a scFv, or a single-chain variable region, e.g., of shark or camelid origin.

In a multimeric binding molecule as provided herein at least three, at least four, at least five, at least six, at least seven, at least eight, at least nine, at least ten, at least eleven or twelve binding unit-associated antigen-binding domains of the binding molecule specifically bind to the same predetermined target on the surface of a cell, e.g., to the same target antigen expressed on the cell, or to the same epitope on the target antigen. In certain embodiments the at least three, at least four, at least five, at least six, at least seven, at least eight, at least nine, at least ten, at least eleven or twelve binding unit-associated antigen-binding domains are identical. In certain embodiments the target is a tumor-specific antigen or a tumor associated antigen. A multimeric binding molecule as provided herein preferentially or selectively binds to a cell, e.g., a diseased cell, e.g., a tumor or other cancer cell, that expresses the predetermined target at a higher density relative to a cell expressing the predetermined target at a lower density, such as a normal healthy cell.

As used herein, “a cell that expresses a predetermined target at higher density relative to a cell expressing the predetermined target at a lower density” refers to, e.g., a cell that expresses a greater number of copies of the target, e.g., a target antigen, on the surface of the cell than a reference cell, or a cell that expresses the target, e.g., a target antigen, in rafts or clusters such that the target antigen is clustered more tightly together than the same target antigen on a reference cell. In certain embodiments the “cell that expresses the predetermined target at a higher density” is a diseased cell, e.g., a cancer cell or tumor cell, and the reference cell “expressing the predetermined target at a lower density” is a normal healthy cell. In certain embodiments the “predetermined target” is an antigen that is overexpressed in response to a disease state of a cell. In certain embodiments the predetermined target is a tumor-specific antigen or a tumor-associated antigen.

In certain embodiments the “predetermined target” can be a TNF receptor superfamily (TNFrSF) target overexpressed on certain immune cells, e.g., activated CD4+ and CD8+ T cells, or regulatory T cells (Treg). In humans, for example the TNFrSF members OX40 and GITR are expressed on activated CD4+ and CD8+ T cells and activated Tregs, but not on most resting naïve or memory T cells. Signaling through OX40 or GITR expressed on CD4+ and CD8+ effector T cells provides enhanced costimulatory proliferation, survival, and effector functions (Id., Stüber E, et al., Immunity 2:507-21 (1995); Tone M, et al., Proc Natl Acad Sci USA. 100:15059-15064 (2003); Ronchetti, S., et al., Eur J Immunol. 34:613-622 (2004)). Given the proper cytokine milieu, OX40 or GITR signaling can also block the immunosuppressive abilities of Tregs, thereby enhancing cytotoxic T lymphocyte (CTL) function (Linch, S N, et al. Front. Oncol. 5:doi: 10.3389/fonc.2015.00034 (2015); Shimizu, J., et al., Nature Immunol 3:135-142 (2002)).

In certain embodiments, the “predetermined target” can be an immune checkpoint molecule, for example, CTLA4, differentially expressed on immune cells.

Antigen density of any given cell can be measured and expressed in various ways. For example, the cells can be treated with an antibody bound to a fluorescent tag, and then subjected to fluorescence-activated cell sorting, or FACS, and the relative level of target antigen expressed on the cells can be reported as a relative mean fluorescence intensity or MFI. In certain embodiments, the cell that expresses the predetermined target at a higher density can have an MFI at least 0.5×, 1×, 5×, 10×, 50×, 100×, 500×, 1000×, 5000×, 10,000×, 50,000×, or 100,000× greater than the MFI measured for the reference cell expressing the target antigen at a lower density. Relative target density of cells can also be measured by flow cytometry using a kit that includes calibration beads with predetermined amounts of primary antibody bound thereto, e.g., the QIFIKit (DAKO). Using a standard curve provided by the calibration beads, relative antigen density can be expressed as “specific antibody binding capacity” (sABC) per cell.

In certain embodiments, a multimeric binding molecule, e.g., an IgM, IgM-like, IgA, or IgA-like antibody as provided herein can have enhanced selectivity for cells expressing a predetermined target at a relatively higher density due, at least in part, to increased valency of the binding molecule for the predetermined target relative to, e.g., a corresponding bivalent IgG therapeutic monoclonal antibody that binds to the same target, with equivalent and/or identical binding unit-associated antigen-binding domains. For example, a pentameric IgM antibody comprising 10 binding unit-associated antigen-binding domains can more readily recognize and bind to a cell over-expressing a tumor-specific antigen or tumor-associated antigen than a corresponding bivalent IgG antibody comprising just two of the binding unit-associated antigen-binding domains. In certain embodiments, a binding molecule, e.g., an IgM, IgM-like, IgA, or IgA-like antibody as provided herein can be engineered such that it binds only weakly or does not detectably bind to cells expressing the predetermined target antigen at lower density, e.g., normal healthy cells, but can detectably bind to cells expressing the target antigen at higher density. In certain embodiments, a binding molecule, e.g., an IgM, IgM-like, IgA, or IgA-like antibody as provided herein can bind to a cell expressing the target antigen at higher density, where a corresponding bivalent IgG antibody having just two equivalent or identical binding unit-associated antigen-binding domains that specifically bind to the predetermined target on the surface of the cell cannot bind to the cell expressing the target antigen at higher density, or to a cell expressing the target antigen at lower density.

In certain embodiments, a binding molecule, e.g., an IgM, IgM-like, IgA, or IgA-like antibody as provided herein binds with enhanced selectivity to cells expressing a predetermined target antigen at higher density through a combination of increased valency, e.g. by virtue of being an IgM, IgM-like, IgA, or IgA-like antibody, and reduced affinity of each binding unit-associated antigen-binding domain for the predetermined target antigen. For example, a binding molecule, e.g., an IgM or IgM-like antibody comprising 10 binding unit-associated antigen-binding domains with low binding affinity for the target antigen, can selectively target only those cells expressing the target antigen at high density due to their increased avidity for the target. As shown in the Examples section below, the inventors have engineered the binding unit-associated antigen-binding domains of a known therapeutic antibody that binds to the B cell marker CD20 such that the binding unit-associated antigen-binding domains have reduced affinity for the target. These reduced affinity binding unit-associated antigen-binding domains, when incorporated into a bivalent IgG background, are incapable of binding to and inducing complement-mediated cytotoxicity (CDC) in any B cell lines, but the same reduced-affinity antigen-binding domains, when incorporated into an IgM background, do not detectably bind to or induce CDC in B cell lines expressing CD20 at low density, but do induce CDC of B cell lines expressing CD20 at high density. Likewise, these reduced affinity binding unit-associated antigen-binding domains, when incorporated into a bispecific IgM background, where the IgM antibody further comprises a modified J-chain comprising a J-chain-associated antigen-binding domain that specifically binds to CD3ε, do not induce T-cell directed cytotoxicity (TDCC) of B cell lines expressing CD20 at low density, but do induce TDCC of B cell lines expressing CD20 at high density. Methods for identifying variants of known therapeutic antibodies that are predicted to bind to the target with reduced affinity are presented elsewhere herein. Methods to make such variants are well known to those in the art.

In certain embodiments, a “cell expressing a predetermined target at higher density” can be a cancer cell or a tumor cell. In certain embodiments the target is a tumor-specific antigen, i.e., a target antigen that is largely expressed only on tumor or cancer cells, or that may be expressed only at undetectable levels in normal healthy cells of an adult. In certain embodiments the target is a tumor-associated antigen, i.e., a target antigen that is expressed on both healthy and cancerous cells but is expressed at much higher density on cancerous cells than on normal healthy cells. Exemplary tumor-specific and tumor-associated antigens include, without limitation, B-cell maturation antigen (BCMA), CD19, CD20, epidermal growth factor receptor (EGFR), human epidermal growth factor receptor 2 (HER2, also called ErbB2), HER3 (ErbB3), receptor tyrosine-protein kinase ErbB4, cytotoxic T-lymphocyte antigen 4 (CTLA4), programmed cell death protein 1 (PD-1), Programmed death-ligand 1 (PD-L1), vascular endothelial growth factor (VEGF), VEGF receptor-1 (VEGFR1), VEGFR2, CD52, CD30, prostate-specific membrane antigen (PSMA), CD38, ganglioside GD2, self-ligand receptor of the signaling lymphocytic activation molecule family member 7 (SLAMF7), platelet-derived growth factor receptor A (PDGFRA), CD22, FLT3 (CD135), CD123, MUC-16, carcinoembryonic antigen-related cell adhesion molecule 1 (CEACAM-1), mesothelin, tumor-associated calcium signal transducer 2 (Trop-2), glypican-3 (GPC-3), human blood group H type 1 trisaccharide (Globo-H), sialyl Tn antigen (STn antigen), or CD33. The skilled person will understand that these target antigens appear in the literature by a number of different names, but that these well-known therapeutic targets can be easily identified using databases available online, e.g., EXPASY.org.

Other tumor associated and/or tumor-specific antigens include, without limitation: DLL4, Notch1, Notch2, Notch3, Notch4, JAG1, JAG2, c-Met, IGF-1R, Patched, Hedgehog family polypeptides, WNT family polypeptides, FZD1, FZD2, FZD3, FZD4, FZD5, FZD6, FZD7, FZD8, FZD9, FZD10, LRP5, LRP6, IL-6, TNFalpha, IL-23, IL-17, CD80, CD86, CD3, CEA, Muc16, PSCA, CD44, c-Kit, DDR1, DDR2, RSPO1, RSPO2, RSPO3, RSPO4, BMP family polypeptides, BMPR1a, BMPR1b, or a TNF receptor superfamily protein such as TNFR1 (DR1), TNFR2, TNFR1/2, CD40 (p50), Fas (CD95, Apo1, DR2), CD30, 4-1BB (CD137, ILA), TRAILR1 (DR4, Apo2), DR5 (TRAILR2), TRAILR3 (DcR1), TRAILR4 (DcR2), OPG (OCIF), TWEAKR (FN14), LIGHTR (HVEM), DcR3, DR3, EDAR, or XEDAR.

In certain embodiments, the at least three, at least four, at least five, at least six, at least seven, at least eight, at least nine, at least ten, at least eleven or twelve binding unit-associated antigen-binding domains specific for the predetermined target are a reduced-affinity variant of an antigen-binding domain of an existing antibody known to bind to the predetermined target, e.g., an approved therapeutic antibody or a therapeutic antibody in preclinical or clinical development. Exemplary known therapeutic antibodies from which to prepare reduced affinity variants include, without limitation alemtuzumab (binds to CD52), atezolizumab (binds to PD-L1), avelumab (binds to PD-L1), bevacizumab (binds to VEGF), blinatumomab (binds to CD19), brentuximab (binds to CD30), capromab (binds to PSMA), cetuximab (binds to EGFR), daratumumab (binds to CD38), denosumab (binds to RANKL), dinutuximab (binds to GD2), durvalumab (binds to PD-L1), elotuzumab (binds to SLAMF7), gemtuzumab (binds to CD33), ibritumomab (binds to CD20), ipilimumab (binds to CTLA4), inotuzumab (binds to CD22), necitumumab (binds to EGFR), nivolumab (binds to PD-1), obinutuzumab (binds to CD20), ocrelizumab (binds to CD20), ofatumumab (binds to CD20), olaratumab (binds to PDGFRA), omalizumab (binds to IgE), panitumumab (binds to EGFR), pembrolizumab (binds to PD-1), pertuzumab (binds to HER2), ramucirumab (binds to VEGFR2), ranibizumab (binds to VEGFR1 and VEGFR2), rituximab (binds to CD20), trastuzumab (binds to HER2), or tremelimumab (binds to CTLA4).

In certain embodiments, the reduced-affinity variant of an antigen-binding domain of an existing antibody, including, but not limited to those listed above, binds to the predetermined target with a binding affinity for the predetermined target at least 1-fold, at least 3-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 500-fold, at least 1000-fold, at least 5000-fold, at least 10,000-fold, at least 50,000-fold, or at least 100,000-fold or more lower than binding affinity of the antigen-binding domain of the existing antibody. In other words, reduced-affinity variant binding domains comprise a dissociation constant (K_{D}) that is a higher value than the K_{D} of the existing antibody's antigen-binding domains.

In certain embodiments, the antigen-binding domains of the existing antibody comprises an antibody heavy chain variable region (VH) and an antibody light chain variable region (VL) comprising, respectively, the VH and VL amino acid sequences of rituximab, SEQ ID NO: 7 and SEQ ID NO: 8, the VH and VL amino acid sequences of the anti CD20 monoclonal antibody 1.5.3, SEQ ID NO: 14 and SEQ ID NO: 15, the VH and VL amino acid sequences of ipilimumab, SEQ ID NO: 16 and SEQ ID NO: 17, the VH and VL amino acid sequences of tremelimumab, SEQ ID NO: 18 and SEQ ID NO: 19, the VH and VL amino acid sequences of trastuzumab, SEQ ID NO: 20 and SEQ ID NO: 21, the VH and VL amino acid sequences of cetuximab, SEQ ID NO: 22 and SEQ ID NO: 23, the VH and VL amino acid sequences of ocrelizumab, SEQ ID NO: 24 and SEQ ID NO: 25, the VH and VL amino acid sequences of obinutuzumab, SEQ ID NO: 26 and SEQ ID NO: 27, the VH and VL amino acid sequences of pertuzumab, SEQ ID NO: 28 and SEQ ID NO: 29, the VH and VL amino acid sequences of omalizumab, SEQ ID NO: 30 and SEQ ID NO: 31, the VH and VL amino acid sequences of pembrolizumab, SEQ ID NO: 32 and SEQ ID NO: 33, the VH and VL amino acid sequences of avelumab, SEQ ID NO: 34 and SEQ ID NO: 35, the VH and VL amino acid sequences of atezolizumab, SEQ ID NO: 36 and SEQ ID NO: 37, the VH and VL amino acid sequences of durvalumab, SEQ ID NO: 38 and SEQ ID NO: 39, or the VH and VL amino acid sequences of nivolumab, SEQ ID NO: 40 and SEQ ID NO: 41. These sequences are provided in Table 2:

TABLE 2
Antibody Sequences

| | | | VH | | VL | |
|---|---|---|---|---|---|---|
| | | | SEQ | | SEQ | |
| | | | ID | | ID | |
| Source | Name | Target | NO: | VH | NO: | VL |
| drugbank.ca | rituximab | CD20 | 7 | QVQLQQPGAELVKPGASVKMSCK | 8 | QIVLSQSPAILSASPGEKVTMTCRASSSV |
| Accession | | | | ASGYTFTSYNMHWVKQTPGRGLE | | SYIHWFQQKPGSSPKPWIYATSNLASGV |
| #DB00073 | | | | WIGAIYPGNGDTSYNQKFKGKATL | | PVRFSGSGSGTSYSLTISRVEAEDAATYY |
| | | | | TADKSSSTAYMQLSSLTSEDSAVY | | CQQWTSNPPTFGGGTKLEIKR |
| | | | | YCARSTYYGGDWYFNVWGAGTT | | |
| | | | | VTVSA | | |
| | Ritux VL N93D | CD20 | | | 9 | QIVLSQSPAILSASPGEKVTMTCRASSSV |
| | | | | | | SYIHWFQQKPGSSPKPWIYATSNLASGV |
| | | | | | | PVRFSGSGSGTSYSLTISRVEAEDAATYY |
| | | | | | | CQQWTSDPPTFGGGTKLEIKR |
| | Ritux VL N93E | CD20 | | | 10 | QIVLSQSPAILSASPGEKVTMTCRASSSV |
| | | | | | | SYIHWFQQKPGSSPKPWIYATSNLASGV |
| | | | | | | PVRFSGSGSGTSYSLTISRVEAEDAATYY |
| | | | | | | CQQWTSEPPTFGGGTKLEIKR |
| | Ritux VL N93A | CD20 | | | 11 | QIVLSQSPAILSASPGEKVTMTCRASSSV |
| | | | | | | SYIHWFQQKPGSSPKPWIYATSNLASGV |
| | | | | | | PVRFSGSGSGTSYSLTISRVEAEDAATYY |
| | | | | | | CQQWTSAPPTFGGGTKLEIKR |
| | Ritux VL N93K | CD20 | | | 12 | QIVLSQSPAILSASPGEKVTMTCRASSSV |
| | | | | | | SYIHWFQQKPGSSPKPWIYATSNLASGV |
| | | | | | | PVRFSGSGSGTSYSLTISRVEAEDAATYY |
| | | | | | | CQQWTSKPPTFGGGTKLEIKR |
| | Ritux VL N93R | CD20 | | | 13 | QIVLSQSPAILSASPGEKVTMTCRASSSV |
| | | | | | | SYIHWFQQKPGSSPKPWIYATSNLASGV |
| | | | | | | PVRFSGSGSGTSYSLTISRVEAEDAATYY |
| | | | | | | CQQWTSRPPTFGGGTKLEIKR |
| US2007001 | 1.5.3 | CD20 | 14 | EVQLVQSGAEVKKPGESLKISCKG | 15 | DIVMTQTPLSSPVTLGQPASISCRSSQSL |
| 4720 | | | | SGYSFTSYWIGWVRQMPGKGLEW | | VYSDGNTYLSWLQQRPGQPPRLLIYKIS |
| | | | | MGIIYPGDSDTRYSPSFQGQVTISA | | NRFSGVPDRFSGSGAGTDFTLKISRVEA |
| | | | | DKSITTAYLQWSSLKASDTAMYYC | | EDVGVYYCVQATQFPLTFGGGTKVEIK |
| | | | | ARHPSYGSGSPNFDYWGQGTLVTV | | |
| | | | | SS | | |
| US2015028 | ipilimumab | CTLA4 | 16 | QVQLVESGGGVVQPGRSLRLSCAA | 17 | EIVLTQSPGTLSLSPGERATLSCRASQSV |
| 3234 | | | | SGFTFSSYTMHWVRQAPGKGLEW | | GSSYLAWYQQKPGQAPRLLIYGAFSRA |
| | | | | VTFISYDGNNKYYADSVKGRFTISR | | TGIPDRFSGSGSGTDFTLTISRLEPEDFAV |
| | | | | DNSKNTLYLQMNSLRAEDTAIYYC | | YYCQQYGSSPWTFGQGTKVEIK |
| | | | | ARTGWLGPFDYWGQGTLVTVSS | | |
| U.S. Pat. | tremelimumab | CTLA4 | 18 | QVQLVESGGGVVQPGRSLRLSCAA | 19 | DIQMTQSPSSLSASVGDRVTITCRASQSI |
| No. | | | | SGFTFSSYGMHWVRQAPGKGLEW | | NSYLDWYQQKPGKAPKLLIYAASSLQS |
| 8,491,895 | | | | VAVIWYDGSNKYYADSVKGRFTIS | | GVPSRFSGSGSGTDFTLTISSLQPEDFAT |
| B2 | | | | RDNSKNTLYLQMNSLRAEDTAVY | | YYCQQYYSTPFTFGPGTKVEIK |
| | | | | YCARDPRGATLYYYYYGMDVWG | | |
| | | | | QGTTVTVSS | | |
| U.S. Pat. | trastuzumab | HER2 | 20 | EVQLVESGGGLVQPGGSLRLSCAA | 21 | DIQMTQSPSSLSASVGDRVTITCRASQD |
| No. | | | | SGFNIKDTYIHWVRQAPGKGLEWV | | VNTAVAWYQQKPGKAPKLLIYSASFLY |
| 5,821,337 | | | | ARIYPTNGYTRYADSVKGRFTISAD | | SGVPSRFSGSRSGTDFTLTISSLQPEDFAT |
| | | | | TSKNTAYLQMNSLRAEDTAVYYC | | YYCQQHYTTPPTFGQGTKVEIK |
| | | | | SRWGGDGFYAMDYWGQGTLVTV | | |
| | | | | SS | | |
| drugbank.ca | Cetuximab | EGFR | 22 | QVQLKQSGPGLVQPSQSLSITCTVS | 23 | DILLTQSPVILSVSPGERVSFSCRASQSIG |
| Accession | | | | GFSLTNYGVHWVRQSPGKGLEWL | | TNIHWYQQRTNGSPRLLIKYASESISGIP |
| #DB00002 | | | | GVIWSGGNTDYNTPFTSRLSINKDN | | SRFSGSGSGTDFTLSINSVESEDIADYYC |
| | | | | SKSQVFFKMNSLQSNDTAIYYCAR | | QQNNNWPTTFGAGTKLELK |
| | | | | ALTYYDYEFAYWGQGTLVTVSA | | |
| U.S. Pat. | ocrelizumab | CD20 | 24 | EVQLVESGGGLVQPGGSLRLSCAA | 25 | DIQMTQSPSSLSASVGDRVTITCRASSSV |
| No. | | | | SGYTFTSYNMHWVRQAPGKGLEW | | SYMHWYQQKPGKAPKPLIYAPSNLASG |
| 8,679,767 | | | | VGAIYPGNGDTSYNQKFKGRFTISV | | VPSRFSGSGSGTDFTLTISSLQPEDFATY |
| | | | | DKSKNTLYLQMNSLRAEDTAVYY | | YCQQWSFNPPTFGQGTKVEIK |
| | | | | CARVVYYSNSYWYFDVWGQGTL | | |
| | | | | VTVSS | | |
| US2014024 | obinutuzumab | CD20 | 26 | QVQLVQSGAEVKKPGSSVKVSCK | 27 | DIVMTQTPLSLPVTPGEPASISCRSSKSLL |
| 8262A1 | | | | ASGYAFSYSWINWVRQAPGQGLE | | HSNGITYLYWYLQKPGQSPQLLIYQMSN |
| | | | | WMGRIFPGDGDTDYNGKFKGRVTI | | LVSGVPDRFSGSGSGTDFTLKISRVEAED |
| | | | | TADKSTSTAYMELSSLRSEDTAVY | | VGVYYCAQNLELPYTFGGGTKVEIKRT |
| | | | | YCARNVFDGYWLVYWGQGTLVT | | V |
| | | | | VSS | | |
| drugbank.ca | pertuzumab | Her2 | 28 | EVQLVESGGGLVQPGGSLRLSCAA | 29 | DIQMTQSPSSLSASVGDRVTITCKASQD |
| Accession | | | | SGFTFTDYTMDWVRQAPGKGLEW | | VSIGVAWYQQKPGKAPKLLIYSASYRYT |
| #DB06366 | | | | VADVNPNSGGSIYNQRFKGRFTLS | | GVPSRFSGSGSGTDFTLTISSLQPEDFAT |
| | | | | VDRSKNTLYLQMNSLRAEDTAVY | | YYCQQYYIYPYTFGQGTKVEIK |
| | | | | YCARNLGPSFYFDYWGQGTLVTVS | | |
| | | | | S | | |
| drugbank.ca | omalizumab | Human | 30 | EVQLVESGGGLVQPGGSLRLSCAV | 31 | DIQLTQSPSSLSASVGDRVTITCRASQSV |
| Accession | | IgE | | SGYSITSGYSWNWIRQAPGKGLEW | | DYDGDSYMNWYQQKPGKAPKLLIYAA |
| #DB00043 | | | | VASITYDGSTNYADSVKGRFTISRD | | SYLESGVPSRFSGSGSGTDFTLTISSLQPE |
| | | | | DSKNTFYLQMNSLRAEDTAVYYC | | DFATYYCQQSHEDPYTFGQGTKVEIK |
| | | | | ARGSHYFGHWHFAVWGQGTLVTV | | |
| | | | | SS | | |
| drugbank.ca | pembrolizumab | PD-1 | 32 | QVQLVQSGVEVKKPGASVKVSCK | 33 | EIVLTQSPATLSLSPGERATLSCRASKGV |
| Accession | | | | ASGYTFTNYYMYWVRQAPGQGLE | | STSGYSYLHWYQQKPGQAPRLLIYLASY |
| #DB09037 | | | | WMGGINPSNGGTNFNEKFKNRVTL | | LESGVPARFSGSGSGTDFTLTISSLEPEDF |
| | | | | TTDSSTTTAYMELKSLQFDDTAVY | | AVYYCQHSRDLPLTFGGGTKVEIK |
| | | | | YCARRDYRFDMGFDYWGQGTTVT | | |
| | | | | VSS | | |
| drugbank.ca | avelumab | PD-L1 | 34 | EVQLLESGGGLVQPGGSLRLSCAA | 35 | QSALTQPASVSGSPGQSITISCTGTSSDV |
| Accession | | | | SGFTFSSYIMMWVRQAPGKGLEW | | GGYNYVSWYQQHPGKAPKLMIYDVSN |
| #DB 11945 | | | | VSSIYPSGGITFYADTVKGRFTISRD | | RPSGVSNRFSGSKSGNTASLTISGLQAED |
| | | | | NSKNTLYLQMNSLRAEDTAVYYC | | EADYYCSSYTSSSTRVFGTGTKVTVL |
| | | | | ARIKLGTVTTVDYWGQGTLVTVSS | | |
| drugbank.ca | atezolizumab | PD-L1 | 36 | EVQLVESGGGLVQPGGSLRLSCAA | 37 | DIQMTQSPSSLSASVGDRVTITCRASQD |
| Accession | | | | SGFTFSDSWIHWVRQAPGKGLEW | | VSTAVAWYQQKPGKAPKLLIYSASFLYS |
| #DB11595 | | | | VAWISPYGGSTYYADSVKGRFTIS | | GVPSRFSGSGSGTDFTLTISSLQPEDFAT |
| | | | | ADTSKNTAYLQMNSLRAEDTAVY | | YYCQQYLYHPATFGQGTKVEIK |
| | | | | YCARRHWPGGFDYWGQGTLVTVS | | |
| | | | | S | | |
| drugbank.ca | durvalumab | PD-L1 | 38 | EVQLVESGGGLVQPGGSLRLSCAA | 39 | EIVLTQSPGTLSLSPGERATLSCRASQRV |
| Accession | | | | SGFTFSRYWMSWVRQAPGKGLEW | | SSSYLAWYQQKPGQAPRLLIYDASSRAT |
| #11714 | | | | VANIKQDGSEKYYVDSVKGRFTIS | | GIPDRFSGSGSGTDFTLTISRLEPEDFAV |
| | | | | RDNAKNSLYLQMNSLRAEDTAVY | | YYCQQYGSLPWTFGQGTKVEIK |
| | | | | YCAREGGWFGELAFDYWGQGTLV | | |
| | | | | TVSS | | |
| drugbank.ca | nivolumab | PD-1 | 40 | QVQLVESGGGVVQPGRSLRLDCK | 41 | EIVLTQSPATLSLSPGERATLSCRASQSV |
| Accession | | | | ASGITFSNSGMHWVRQAPGKGLE | | SSYLAWYQQKPGQAPRLLIYDASNRAT |
| #DB09035 | | | | WVAVIWYDGSKRYYADSVKGRFT | | GIPARFSGSGSGTDFTLTISSLEPEDFAVY |
| | | | | ISRDNSKNTLFLQMNSLRAEDTAV | | YCQQSSNWPRTFGQGTKVEIK |
| | | | | YYCATNDDYWGQGTLVTVSS | | |

In certain embodiments, the antigen-binding domains of the existing antibody comprises an antibody heavy chain variable region (VH) and an antibody light chain variable region (VL) comprising, respectively, the VH and VL amino acid sequences of rituximab, SEQ ID NO: 7 and SEQ ID NO: 8. The inventors performed in silico modeling of the VH and VL amino acid sequences of rituximab in conjunction with the known crystal structure of the rituximab Fab bound to the CD20 protein to identify various amino acid substitutions that were predicted to either increase or decrease the binding affinity of antigen-binding domain for its epitope on CD20. Various protein modeling and prediction algorithms can be used to predict antibody binding characteristics including, but not limited to BioLuminate (Schrodinger), Lasergene Structural Biology Suite (DNASTAR), Immunoinformatics (OMIC Tools), Biovia (Dassault Systems), and RosettaAntibody (Rosetta Software). Alternatively, VH and VL sequences of known antibodies can be subjected to shotgun mutagenesis by standard methods using, e.g., alanine scanning, and the resulting antigen-binding domains can be tested for reduced affinity binding to the predetermined target antigen of interest.

Using the BioLuminate tools, the inventors identified and constructed several exemplary variants of the rituximab antigen-binding domain with amino acid substitutions in the VL of rituximab (SEQ ID NO: 8) at position N93 that were predicted to result in modified binding affinity. Several substitutions at position N93 of the rituximab light chain variable region were ranked according to their predicted effect on binding affinity (N93D <N93E<N93A<WT<N93K<N93R). DNA fragments encoding these mutant light chain variable regions RTX N93D (SEQ ID NO: 9), RTX N93E (SEQ ID NO: 10), RTX N93A (SEQ ID NO: 11), RTX N93K (SEQ ID NO: 12), and RTX N93R (SEQ ID NO: 13), were synthesized, as described in the Examples below. Of these, variant antigen-binding domains with the N93D and N93E substitutions, as predicted, showed reduced affinity for CD20 relative to the wild type rituximab antigen-binding domain. IgM antibodies comprising the variant binding unit-associated antigen-binding domains could direct complement-mediated (CDC) killing of a B cell line expressing CD20 at high density but did not direct CDC of a B cell line expressing CD20 at low density. IgG antibodies comprising the variant antigen-binding domains failed to bind to the high density CD20 expressing cell line and failed to elicit CDC in either the high or low density CD20 expressing B cell lines.

In certain embodiments, e.g., where it is desired to test antigen-binding domains for their binding characteristics to cells expressing the predetermined target of interest at higher and lower density, various cell lines can be employed. For example, if CD20 is the predetermined target of interest, high and low density CD20-expressing cell lines, e.g., B-cell lymphoma cell lines are available, and can be graded for their level of CD20 expression by various methods, some of which are provided elsewhere herein. Cell lines expressing CD20 at high density include, without limitation, the Ramos cell line, the Raji cell line, the DoHH-2 cell line, the JeKo-1 cell line, the Z-138 cell line, the Daudi cell line, the Granta cell line, or the DoHH2 cell line. Cell lines expressing CD20 at lower density include, without limitation, the CA46 cell line, the Nalm-1 cell line, the Toledo cell line, the BJAB cell line, the Kasumi-2 cell line, the RPMI 8226 cell line, the HT cell line, the SU-DHL-8 cell line, the JM1 cell line, the Namalwa cell line, the Nalm-6 cell line, or the Z138 cell line.

In certain embodiments, a multimeric anti-CD20 binding molecule as provided herein, e.g., an IgA, IgA-like, IgM, or IgM-like antibody comprising at least three, at least four, at least five, at least six, at least seven, at least eight, at least nine, at least ten, at least eleven or twelve binding unit-associated antigen-binding domains comprising a VH with the amino acid sequence SEQ ID NO: 7 and a VL with the amino acid sequence SEQ ID NO: 9 or SEQ ID NO: 10 can direct complement-mediated killing of Ramos cells, wherein an equivalent amount of a monospecific bivalent IgG1 antibody comprising two of the same antigen-binding domains shows no detectable complement-mediated killing of Ramos cells. Moreover in certain embodiments, a multimeric anti-CD20 binding molecule as provided herein, e.g., an IgA, IgA-like, IgM, or IgM-like antibody comprising at least three, at least four, at least five, at least six, at least seven, at least eight, at least nine, at least ten, at least eleven or twelve binding unit-associated antigen-binding domains comprising a VH with the amino acid sequence SEQ ID NO: 7 and a VL with the amino acid sequence SEQ ID NO: 9 or SEQ ID NO: 10 can direct complement-mediated (CDC), T-cell-mediated (if the binding molecule is bispecific and comprises, for example, a modified J-chain that binds to CD3ε as described elsewhere herein), or both complement-mediated and T-cell-mediated killing of CD20-positive malignant B cells in a subject with cancer, but either minimally affects or does not affect normal B cells in the subject with cancer. In certain embodiments the cancer is a high-expressing CD20-positive leukemia, lymphoma, or myeloma. In certain embodiments, the subject is a human subject.

Reduced affinity variants of other known therapeutic antibodies, including, but not limited to those described herein can be likewise predicted, constructed, and tested by the methods described herein. For example, similar modeling of two CTLA4 antibodies, ipilimumab (VH: SEQ ID NO: 16 and VL: SEQ ID NO: 17 and tremelimumab (VH: SEQ ID NO: 18 and VL: SEQ ID NO: 19) was carried out using the BioLuminate software in conjunction with the known crystal structures of Fabs of the antigen-binding domains of ipilimumab and tremelimumab. The modeling predicted the following substitutions as potentially lowering the binding affinity of these antigen-binding domains for their target, CTLA4.

For ipilimumab, amino acid substitutions predicted to reduce binding affinity of the antigen-binding domain for CTLA4 include, but are not limited to, heavy chain substitutions at F50 of SEQ ID NO: 16, e.g., F50D, F50N, F50H, or F50E; heavy chain substitutions at S52 of SEQ ID NO: 16, e.g., S52H, S52D, S52K, S521, S52T, S52F, S52N, S52W, or S52Y; heavy chain substitutions at Y53 of SEQ ID NO: 16, e.g., Y53S, Y53Q, Y52K, Y53T, Y53F, Y53N, Y53H, Y53W, Y53V, Y53E, or Y53L; heavy chain substitutions at N56 of SEQ ID NO: 16, e.g., N56D; heavy chain substitutions at N57 of SEQ ID NO: 16, e.g., N57D, N57S, N57G, or N57E; heavy chain substitutions at Y59 of SEQ ID NO: 16, e.g., Y59H, Y59K, Y591, Y59F, Y59R, Y59W, or Y59L; light chain substitutions at G93 of SEQ ID NO: 17, e.g., G93E; light chain substitutions at S95 of SEQ ID NO: 17, e.g., S95H, S95K, S95N, S95G, or S95A; or light chain substitutions at W97 of SEQ ID NO: 17, e.g., W97D or W97E.

For tremelimumab, amino acid substitutions predicted to reduce binding affinity of the antigen-binding domain for CTLA4 include, but are not limited to, heavy chain substitutions at W52 of SEQ ID NO: 18, e.g., W52Q, W52G, W52E. W52C, W52D, W52S, W52P, W52H, W52N, W52V, W52T, or W52A; heavy chain substitutions at N57 of SEQ ID NO: 18, e.g., N57D; any heavy chain substitutions at R101 of SEQ ID NO: 18; heavy chain substitutions at G102 of SEQ ID NO: 18, e.g., G102Q, G102D, or G102F; heavy chain substitutions at Y106 of SEQ ID NO: 18, e.g., Y106G, Y106E, or Y106D; heavy chain substitutions at Y107 of SEQ ID NO: 18, e.g., Y107Q, Y107C, Y107K, Y107P, Y107H, Y107N, Y107V, Y107T, Y107A, or Y107L; heavy chain substitutions at Y110 of SEQ ID NO: 18, e.g., Y110I, Y110Q, Y110G, Y110E, Y110C, Y110D, Y110S, Y110K, Y110P, Y110N, Y110H, Y110V, Y110T, Y110W, or Y110A; light chain substitutions at S28 of SEQ ID NO: 19, e.g., S28H or S28R; light chain substitutions at 129 of SEQ ID NO: 19, e.g., I29W; light chain substitutions at N30 of SEQ ID NO: 19, e.g., N30I, N30G, N30E, N30C, N30D, N30S, N30K, N30H, N30V, N30T, N30F, N30A, or N30L; or light chain substitutions at Y32 of SEQ ID NO: 19, e.g., Y32I, Y32Q, Y32C, Y32K, Y32P, Y32H, Y32N, Y32V, Y32T, or Y32L.

In certain embodiments, the at least four, at least five, at least six, at least seven, at least eight, at least nine, at least ten, at least eleven, or twelve antigen-binding domains of a binding molecule, e.g., an antibody as provided herein are identical and specifically bind to the predetermined target on the surface of a cell.

In certain embodiments the binding molecule is a dimeric antibody, e.g., a tetravalent IgA or IgA-like antibody, comprising two bivalent IgA binding units and a J-chain or functional fragment or variant thereof, wherein each binding unit comprises two IgA or IgA-like heavy chain constant regions or multimerizing fragments thereof comprising at least a Cα3 domain and a tailpiece (tp) domain, e.g., comprising Cα3 domain and a tp domain as well as a Cα1 domain, an IgA hinge region, and/or a Cα2 domain, each associated with a binding unit-associated antigen-binding domain, e.g., a reduced affinity antigen-binding domain as provided herein. The antibody can further comprise a secretory component, or fragment or variant thereof.

In certain embodiments, the binding molecule is a pentameric or a hexameric IgM or IgM-like antibody comprising five or six bivalent IgM binding units, respectively, where each binding unit comprises two IgM or IgM-like heavy chain constant regions or multimerizing fragments thereof comprising at least a Cμ4 domain and a tp domain, e.g., comprising a Cμ4 domain and a tp domain as well as a Cμ2 domain, a Cp domain, and/or a Cμ3 domain, each associated with a binding unit-associated antigen-binding domain, e.g., a reduced affinity antigen-binding domain as provided herein. In certain embodiments the IgM or IgM-like antibody is pentameric, and further comprises a J-chain, or functional fragment or variant thereof. In certain embodiments the IgM or IgM-like heavy chain constant regions can be modified to modulate, e.g., reduce or block, the binding molecule's ability to facilitate complement-dependent cellular cytotoxicity (CDC). In some embodiments, the IgM or IgM-like heavy chain constant regions can be modified to increase serum half-life of the binding molecule

In certain embodiments a multimeric binding molecule, e.g., an IgM, IgM-like, IgA, or IgA-like antibody as provided herein comprises a J-chain or functional fragment or variant thereof, e.g., a human J-chain comprising the amino acid sequence SEQ ID NO: 42 or a functional fragment thereof or a functional variant thereof, e.g., variant of SEQ ID NO: 42 with an amino acid substitution at position 102 (Y102A) that increases serum half-life of an IgM pentameric antibody comprising the variant J-chain (e.g., SEQ ID NO: 43). In certain embodiments, the J-chain or fragment thereof is a modified J-chain further comprising one or more heterologous polypeptides, wherein the heterologous polypeptides are directly or indirectly fused to the J-chain or fragment thereof, e.g., via a peptide linker. In certain embodiments the modified J-chain is a modified human J-chain that specifically binds to CD3R, e.g., SEQ ID NO: 46, SEQ ID NO: 47, or SEQ ID NO: 56.

Polynucleotides, Vectors, and Host Cells

The disclosure further provides a polynucleotide, e.g., an isolated, recombinant, and/or non-naturally-occurring polynucleotide, comprising a nucleic acid sequence that encodes a polypeptide subunit of a multimeric binding molecule, e.g., an IgM, IgM-like, IgA, or IgA-like antibody as provided herein. By “polypeptide subunit” is meant a portion of an antibody, binding molecule, binding unit, or binding domain that can be independently translated. Examples include, without limitation, an antibody VH, an antibody VL, a single chain Fv, an antibody heavy chain, an antibody light chain, an antibody heavy chain constant region, an antibody light chain constant region, a J-chain, a secretory component, and/or any functional (e.g., antigen-binding and/or multimerizing) fragment thereof.

The disclosure further provides a composition comprising two or more polynucleotides, where the two or more polynucleotides collectively can encode a multimeric binding molecule, e.g., an IgM, IgM-like, IgA, or IgA-like antibody as provided herein. In certain embodiments the composition can include a polynucleotide encoding an IgM, IgM-like, IgA, or IgA-like heavy chain or fragment thereof, e.g., a human IgM, IgM-like, IgA, or IgA-like heavy chain as described above where the IgM, IgM-like, IgA, or IgA-like heavy chain comprises at least the VH of a binding unit-associated antigen-binding domain that binds to a predetermined target, e.g., a tumor-specific antigen or a tumor-associated antigen, where the binding molecule, e.g., an IgM, IgM-like, IgA, or IgA-like antibody, selectively binds to cells expressing the predetermined target at higher density than a reference cell, e.g., a normal healthy cell, that expresses the predetermined target at lower density, and a polynucleotide encoding a light chain or fragment thereof, e.g., a human kappa or lambda light chain that comprises at least the VL of a binding unit-associated antigen-binding domain that binds to a predetermined target, e.g., a tumor-specific antigen or a tumor-associated antigen, where the binding molecule, e.g., an IgM, IgM-like, IgA, or IgA-like antibody, selectively binds to cells expressing the predetermined target at higher density than a reference cell, e.g., a normal healthy cell, that expresses the predetermined target at lower density. A polynucleotide composition as provided can further include a polynucleotide encoding a J-chain, e.g., a human J-chain, or a fragment thereof or a variant thereof. In certain embodiments the polynucleotides making up a composition as provided herein can be situated on two or three separate vectors, e.g., expression vectors. Such vectors are provided by the disclosure. In certain embodiments two or more of the polynucleotides making up a composition as provided herein can be situated on a single vector, e.g., an expression vector. Such a vector is provided by the disclosure.

The disclosure further provides a host cell, e.g., a prokaryotic or eukaryotic host cell, comprising a polynucleotide or two or more polynucleotides, encoding a multimeric binding molecule, e.g., an IgM, IgM-like, IgA, or IgA-like antibody as provided herein, or any subunit thereof, a polynucleotide composition as provided herein, or a vector or two, three, or more vectors that collectively encode a multimeric binding molecule, e.g., an IgM, IgM-like, IgA, or IgA-like antibody as provided herein, or any subunit thereof. In certain embodiments a host cell provided by the disclosure can express a multimeric binding molecule, e.g., an IgM, IgM-like, IgA, or IgA-like antibody as provided herein, or a subunit thereof.

In a related embodiment, the disclosure provides a method of producing a multimeric binding molecule, e.g., an IgM, IgM-like, IgA, or IgA-like antibody as provided herein, where the method comprises culturing a host cell as described above and recovering the binding molecule, e.g., the IgM, IgM-like, IgA, or IgA-like antibody.

Methods of Use

This disclosure provides methods for treating cell-based diseases, e.g., cancer, using a multimeric binding molecule, e.g., an IgM, IgM-like, IgA, or IgA-like antibody as provided herein, where the binding molecule, e.g., IgM, IgM-like, IgA, or IgA-like antibody, selectively binds to cells expressing a predetermined target antigen of interest at higher density than that of a reference cell, e.g., a normal healthy cell, that expresses the target antigen and lower density. This disclosure further provides for the use of a multimeric binding molecule, e.g., an IgM, IgM-like, IgA, or IgA-like antibody as provided herein in the preparation of a medicament for treating cell-based diseases, e.g., cancer, where the binding molecule, e.g., IgM, IgM-like, IgA, or IgA-like antibody, selectively binds to cells expressing a predetermined target antigen of interest at higher density than that of a reference cell, e.g., a normal healthy cell, that expresses the target antigen and lower density. This disclosure further provides a multimeric binding molecule, e.g., an IgM, IgM-like, IgA, or IgA-like antibody as provided herein for treating cell-based diseases, e.g., cancer, where the binding molecule, e.g., IgM, IgM-like, IgA, or IgA-like antibody, selectively binds to cells expressing a predetermined target antigen of interest at higher density than that of a reference cell, e.g., a normal healthy cell, that expresses the target antigen and lower density. The methods described below apply equally to use of the provided compositions in the preparation of a medicament for treating disease, and to the provided compositions for treating disease.

The methods of treatment provided herein utilize binding molecules, e.g., IgM, IgM-like, IgA, or IgA-like antibodies comprising three or more, at least four, at least five, at least six, at least seven, at least eight, at least nine, at least ten, at least eleven, or twelve antigen-binding domains, e.g., reduced affinity antigen-binding domains, derived, e.g., from existing therapeutic antibodies such as, but not limited to those described herein, including without limitation reduced-affinity variants of the antibodies comprising the VH and VL amino acid sequences provided in Table 2, or antigen-binding variants, derivatives, or analogs thereof. In certain embodiments the multimeric binding molecule, e.g., the IgM, IgM-like, IgA, or IgA-like antibody can provide enhanced selectivity for cells expressing the predetermined target antigen at higher density, e.g., cancer cells. Based on this disclosure, construction of a multimeric IgA- or IgM-based binding molecule, e.g., IgM, IgM-like, IgA, or IgA-like antibodies with enhanced selectivity for cells expressing a predetermined target antigen at higher density is well within the capabilities of a person of ordinary skill in the art. The improved selectivity can, for example, improve safety and prevent side effects relative to existing therapies, since normal healthy cells can be spared.

In yet another embodiment a multimeric binding molecule, e.g., an IgM, IgM-like, IgA, or IgA-like antibody with enhanced selectivity for cancer cells as provided herein can facilitate cancer treatment, e.g., by slowing tumor growth, stalling tumor growth, or reducing the size of existing tumors, when administered as an effective dose to a subject in need of cancer treatment. The disclosure provides a method of treating cancer comprising administering to a subject in need of treatment an effective dose of a multimeric binding molecule, e.g., an IgM, IgM-like, IgA, or IgA-like antibody as provided herein.

The terms “cancer”, “tumor”, “cancerous”, and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias.

In certain embodiments of the provided method, the cancer to be treated can be a solid tumor, a hematological malignancy, any metastasis thereof, or any combination thereof. In certain embodiments, the solid tumor can be, e.g., a sarcoma, a carcinoma, a melanoma, a lymphoma, any metastases thereof, or any combination thereof. More specifically, the solid tumor can be, e.g., squamous cell carcinoma, adenocarcinoma, basal cell carcinoma, renal cell carcinoma, ductal carcinoma of the breast, soft tissue sarcoma, osteosarcoma, melanoma, small-cell lung cancer, non-small cell lung cancer, adenocarcinoma of the lung, cancer of the peritoneum, hepatocellular carcinoma, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, any metastases thereof, or any combination thereof.

In certain embodiments of the provided method, the cancer to be treated can be a hematologic malignancy or metastasis thereof. In certain embodiments, the hematologic malignancy can be leukemia, lymphoma, myeloma, acute myeloid leukemia, chronic myeloid leukemia, acute lymphocytic leukemia, chronic lymphocytic leukemia, hairy cell leukemia, Hodgkin lymphoma, non-Hodgkin lymphoma, multiple myeloma, any metastases thereof, or any combination thereof.

In certain embodiments, the method provided herein can further include administration of an additional cancer therapy, e.g., surgery, chemotherapy, radiation therapy, a cancer vaccine, or any combination thereof.

This disclosure further provides a method of preventing or treating a cancer in a subject in need thereof, comprising administering to the subject an effective amount of a multimeric binding molecule, e.g., an IgM, IgM-like, IgA, or IgA-like antibody as provided herein, a composition or formulation comprising the binding molecule, e.g., antibody, or a polynucleotide, a vector, or a host cell as described herein.

By “therapeutically effective dose or amount” or “effective amount” is intended an amount of a multimeric binding molecule, e.g., an IgM, IgM-like, IgA, or IgA-like antibody, that when administered brings about a positive immunotherapeutic response with respect to treatment of a cancer patient. In certain embodiments the therapeutically effective dose or amount kills cancer cells but only minimally affects or does not affect, normal cells. In certain embodiments a therapeutically effective amount provides minimal side effects.

Effective doses of compositions for treatment of cancer vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages can be titrated using routine methods known to those of skill in the art to optimize safety and efficacy.

In its simplest form, a preparation to be administered to a subject is a multimeric binding molecule, e.g., an IgM, IgM-like, IgA, or IgA-like antibody as provided herein, administered in conventional dosage form, which can be combined with a pharmaceutical excipient, carrier or diluent as described elsewhere herein.

Pharmaceutical Compositions and Administration Methods

Methods of preparing and administering a multimeric binding molecule, e.g., an IgM, IgM-like, IgA, or IgA-like antibody as provided herein to a subject in need thereof are well known to or are readily determined by those skilled in the art in view of this disclosure. The route of administration of a binding molecule, e.g., an antibody can be, for example, intratumoral, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intratumoral, intravenous, or intraarterial injection or drip. In certain embodiments, a binding molecule, e.g., an IgM, IgM-like, IgA, or IgA-like antibody as provided herein can be introduced locally into a tumor, or in the vicinity of a tumor cell, e.g., within the tumor microenvironment (TME). A suitable pharmaceutical composition can comprise a buffer (e.g. acetate, phosphate, or citrate buffer), a surfactant (e.g. polysorbate), optionally a stabilizer agent (e.g. human albumin), etc.

As discussed herein, a multimeric binding molecule, e.g., an IgM, IgM-like, IgA, or IgA-like antibody as provided herein can be administered in a pharmaceutically effective amount for the in vivo immunotherapeutic treatment of cancers. The disclosed binding molecule, e.g., an IgM, IgM-like, IgA, or IgA-like antibody can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives, and the like. A pharmaceutically effective amount of a multimeric binding molecule, e.g., an IgM, IgM-like, IgA, or IgA-like antibody as provided herein means an amount sufficient to achieve effective binding to a target and to achieve a therapeutic benefit. In certain aspects a pharmaceutically effective amount causes minimal side effects. Suitable formulations are described in Remington: The Science and Practice of Pharmacy (Pharmaceutical Press) 22d ed. (2012).

The amount of a multimeric binding molecule, e.g., an IgM, IgM-like, IgA, or IgA-like antibody that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7^{th} Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9^{th} Edition (Elsevier). Additionally, standard methods in immunology known in the art can be followed, e.g., in Current Protocols in Immunology (Wiley Online Library); Wild, D. (2013), The Immunoassay Handbook 4^{th} Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXAMPLES

### Example 1: Modeling, Construction, and Expression of Altered-Affinity Anti-CD20 IgM Antibody with Enhanced Selectivity Malignant B Cells

Anti-CD20 antibodies, e.g., rituximab, are widely used for treatment of B-cell malignancies, e.g., lymphomas. Unfortunately, CD20 is also broadly expressed on normal B cells, causing undesired B-cell depletion during anti-CD20 cancer therapy. Antigen expression on malignant B cells is typically much higher density than on normal B cells. This Example shows construction and characterization an IgM antibody with anti-CD20 binding domains with reduced affinity. The increased avidity of IgM results in the antibodies selectively targeting those cells that express CD20 at higher density.

Pentameric or hexameric IgM antibodies that can specifically bind to CD20 at reduced affinity relative to rituximab were produced by the following method. The VH (SEQ ID NO: 7) and VL (SEQ ID NO: 8) amino acid sequences of rituximab were subjected to in silico modeling using BioLuminate (Shrodinger) and the available crystal structure of the rituximab Fab and CD20 to predict amino acid substitutions that would result in reduced binding affinity. Several substitutions at position N93 of the rituximab light chain variable region were ranked according to their predicted effect on binding affinity (N93D<N93E<N93A<WT<N93K<N93R). DNA fragments encoding these mutant light variable regions RTX N93D (SEQ ID NO: 9), RTX N93E (SEQ ID NO: 10), RTX N93A (SEQ ID NO: 11), RTX N93K (SEQ ID NO: 12), and RTX N93R (SEQ ID NO: 13), were synthesized along with constructs encoding the wild-type rituximab VH as IgG and IgM antibodies by a commercial vendor.

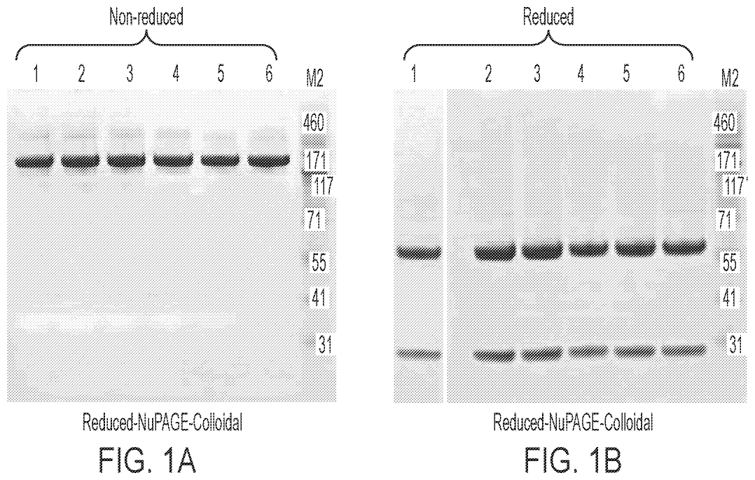

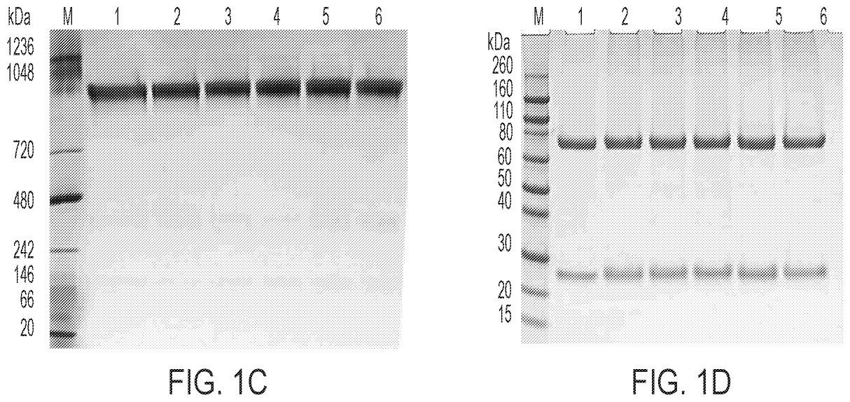

These antibody constructs were expressed and purified as described below The IgG and IgM molecules were resolved on reduced and non-reduced gels as follows. Purified Anti-CD20 IgG and IgM+wild-type J-chain antibodies were analyzed on SDS-PAGE gels under reducing and non-reducing conditions. FIG. 1A depicts a non-reduced gel to resolve assembled IgGs, FIG. 1B depicts a reduced gel to show IgG heavy and light chains, FIG. 1C depicts a non-reduced gel to resolve assembled IgMs, FIG. 1B depicts a reduced gel to show IgM heavy and light chains. The non-reduced IgM gel samples were mixed with NuPage LDS Sample Buffer (Life Technologies #NP0007) and loaded onto a NativePage Novex 3-12% Bis-Tris Gel (Life Technologies #BN1003). Novex Tris-Acetate SDS Running Buffer (Life Technologies #LA0041) was used for gel electrophoresis, and gel was stained with Colloidal Blue Stain (Life Technologies #LC6025). The non-reduced IgG samples were mixed with sample buffer, heated to 80° C. for 10 minutes and loaded on a NuPage Novex 4-12% Bis-Tris Gel (Life Technologies #NP0322). NuPage MES SDS Running Buffer was used for gel electrophoresis and the gel was stained with Colloidal Blue. For the reduced gels, samples were mixed with sample buffer and NuPage reducing agent (Life Technologies #NP0004) and heated to 80° C. for 10 minutes and loaded on a NuPage Novex 4-12% Bis-Tris Gel (Life Technologies #NP0322). NuPage MES SDS Running Buffer (Life Technologies #NP0002) was used for gel electrophoresis and gel was stained with Colloidal Blue.

Protein Expression, Purification, and Characterization

Transfection. IgG or IgM Heavy chain, light chain, and J-chain DNAs (for IgM pentamer constructs) were transfected into, e.g., Expi293 cells. DNA for expression vectors were mixed with Expifectamine and then added to cells. Transient transfection with Expi293 cells was conducted according to the manufacturer's recommendations.

IgG expression products were purified, e.g., using the MabSelectSuRe affinity matrix (GE Life Sciences Catalog #17-5438-01) according to manufacturer's recommendation.

IgM expression products, with or without J-chain were purified, e.g., using the Capture Select IgM affinity matrix (BAC, Thermo Fisher Catalog #2890.05) according to manufacturer's recommendation.

### Example 2: Characterization of CD20 Density on B Cell Lines

An assortment of B cell lines: Raji (ATCC cat. #CCL-86), Ramos (ATCC cat. #CRL-1596), CA46 (ATCC cat. #CRL-1648), DB (ATCC Cat. #CRL-2289), DoHH-2 (DSMZ cat. #ACC-47), JeKo-1 (ATCC cat. #CRL-3006), Z-138 (ATCC cat. #CRL-3001), Toledo (ATCC cat. #CRL-2631), BJAB (DSMZ cat. #ACC-757), Kasumi-2 (DSMZ cat. #ACC-526), Nalm-1 (ATCC cat. #CRL-1567), RPMI 8226 (ATCC cat. #CCL-155), HT (ATCC cat. #CRL-2260), SU-DHL-8 (ATCC cat. #CRL-2961), and JM1 (ATCC cat. #CRL-10423) were tested for relative CD20 surface density using the QIFIKit (DAKO). An unconjugated mouse CD20 antibody (Biolegend) was used at the saturation point, as the primary at 20 μg/mL to allow for monovalent binding. A FITC-conjugated F(ab′)2 antibody (Dako) was used as the secondary at 1:50. Five populations of calibration beads (Dako) bound with a range of defined amounts of primary Mab were mixed and stained in the same manner as cells and set as the standard curve. A FACSCalibur flow cytometer (BD Biosciences) using standard operating procedures was utilized to measure cell surface antigens. FlowJo (Treestar) was used to calculate MFI (Mean Fluorescence Intensity) from each sample. Prism (GraphPad) was used to interpolate antigen density from the standard curve for each cell line. Antigen density expressed as Ab-binding capacity (ABC) in molecules per cell was calculated. Finally, “antigen density” in Table 3 is expressed as the specific ABC (sABC), determined by subtracting the background Ab equivalent of the isotype control from ABC.

The cell lines were also tested for CD20 density by FACS analysis, by the following method. Cells were washed and resuspended in FACS Stain Buffer (FSB, BD cat #554656) at a density of 0.48×10⁶ cells/mL and 25 μL/well was added to a V-bottom 96-well plate. For each well, 5 μL of CD20 antibody (AlexaFluor488 conjugated, Biolegend cat #302316, concentration 200 μg/mL) and 20 μL of CD19 (PE conjugated, BD cat #555413, concentration 0.015 μg/20 μL) were added. For the isotype controls, 5 μL of mouse IgG2b antibody (AlexaFluor488 conjugated, Biolegend cat #400329) and 20 μL of mouse IgG1 antibody (PE conjugated, BD cat #555749) were added. The plate was incubated in the dark and on ice for 30 min. The plate was washed two times with 200 μL of FACS Stain Buffer. The cells were fixed overnight with 50 μL fixing buffer (BD cat #554655 diluted 1:8 in PBS). Data was acquired on a FACSCalibur cytometer and subsequently analyzed using FlowJo (Tree Star).

The various cell lines were also tested for their ability to killed in 10% normal human serum via complement-dependent cytotoxicity by the following method. The B cell lines were maintained in culture in RPMI complete medium (RPMI-C; RPMI 1640 medium, 10 mM HEPES, 1 mM sodium pyruvate, 1×MEM non-essential amino acids, 1× GlutaMAX) containing 10-20% heat-inactivated fetal bovine serum (HI-FBS) (as appropriate for each cell line) at 37° C. in a 5% CO2 incubator. The cells were split every 2-3 days as necessary to maintain the cell density between 0.2-2.0×10⁶ cells/mL. About 5.0×10⁶ cells were recovered by centrifugation and were resuspended in 5.0 mL RPMI-C (1.0×10⁶ cells/mL). Purified anti-CD20 IgG antibody (rituximab) was diluted to 30 μg/mL in RPMI-C, 10% HI FBS, then was serially diluted 3-fold in RPMI-C, 10% HI FBS in a non-tissue culture-treated 96-well plate (Falcon 351177). 10 μL cells (10,000 cells/well), and 10 μL serially diluted Ab were combined in a 384-well plate (Thermo 164610) and incubated for 2 h at 37° C. in a 5% CO₂ incubator. Normal human serum complement (Quidel A113) was diluted to 30% in RPMI-C, 10% HI FBS (1.5 mL:3.5 mL), and then added (10 μL/well 30% NHS or RPMI-C, 10% HI FBS) to each well, and incubated for 4 h at 37° C. in a 5% CO₂ incubator. Cell Titer-Glo reagent (Promega G7572) (10 μL/well) was added to each well and mixed for two minutes on a plate shaker, then for 10 minutes at RT, and read luminescence on PerkinElmer EnVision 2104.

The results are shown in Table 3. The relative CD20 antigen density on cells correlated with MFI and with the ability of the cells to be killed by CDC.

TABLE 3
CD20 Antigen Density on Various B Cell Lines

| | | | CD20 Expression | |
|---|---|---|---|---|
| Cell Line | Disease | N | MFI | Ag Density |
| DB | large cell lymphoma | 1 | 2303 | 818,753 |
| DoHH-2 | B cell lymphoma | 1 | 1388 | 489,911 |
| Raji CR | Burkitt's lymphoma | 1 | 1321 | 465,819 |
| JeKo-1 | mantle cell lymphoma | 1 | 1202 | 422,848 |
| Raji | Burkitt's lymphoma | 1 | 1043 | 365,854 |
| Z-138 | mantle cell lymphoma | 1 | 921 | 321,984 |
| Ramos | Burkitt's lymphoma | 5 | 628 | 257,979 |
| Human B cells | Normal | 4 | 338 | 149,374 |
| Toledo | B cell NHL, diffuse large | 1 | 321 | 106,053 |
| | B cell lymphoma | | | |
| BJAB | Burkitt's lymphoma | 1 | 295 | 96,704 |
| CA46 | Burkitt's lymphoma | 4 | 207 | 95,889 |
| Kasumi-2 | B cell precursor leukemia | 1 | 133 | 38,451 |
| NALM-1 | leukemia | 1 | 108 | 29,461 |
| RPMI 8226 | plasmacytoma, myeloma | 1 | 84 | 20,921 |
| HT | diffuse mixed lymphoma | 1 | 83 | 20,813 |
| SU-DHL-8 | large cell lymphoma | 1 | 64 | 13,801 |
| JM1 | B cell lymphoma | 1 | 54 | 10,223 |

### Example 3: Binding Activity of IgG and IgM Anti-CD20 Mutants on Ramos Cells

The binding affinity of the various IgG and IgM anti-CD20 mutant IgG and IgM antibodies produced in Example 1 on Ramos cells was measured by flow cytometry by the following method. Ramos cells were maintained in culture in RPMI complete medium (RPMI-C; RPMI 1640 medium, 10 mM HEPES, 1 mM sodium pyruvate, 1×MEM non-essential amino acids, 1× GlutaMAX) containing 10% HI-fetal bovine serum at 37° C. in a 5% CO₂ incubator. The cells were split every 2-3 days as necessary to maintain the cell density between 0.2-2.0×10⁶ cells/mL. About 2×10⁶ cells were recovered by centrifugation and were resuspended in 4.0 mL FACS Staining Buffer (FSB, BD 554656; 0.5×10⁶ cells/mL). Purified IgG or IgM antibodies, prepared as described in Example 1, were diluted to 20 μg/mL FBS, then serially diluted 3-fold in FSB in a non-TC-treated 96-well plate (Falcon 351177). Cells (25 μL, 12,500 cells/well) and serially diluted antibody samples (25 μL) were combined in a 96-well, V-bottom plate (Sarstedt 82.1583.001), and incubated for 2 h at 37° C. in a 5% CO2 incubator. The cells were washed twice with 200 μL cold FSB and were resuspended in 50 μL. mouse anti-human kappa antibody AF647 at 1 μg/mL (BioLegend 316514, clone MHK-49, 50 μg/mL; 100 μL antibody:5 mL FSB). The antibody cell mixtures were incubated for 30 minutes on ice in the dark. The cells were again washed and then resuspended in 80 μL FSB (unstained cells) or 80 μL 1% 7-aminoactinomycin D (7-AAD, BD 51-88881E, 1:100 in FSB)(stained cells) and the mixtures were transferred to minitubes (Nova 32022). Fluorescent staining data was acquired on FACSCalibur using standard settings and analyzed with FloJo 10. Mean fluorescent intensity (MFI) values were imported into GraphPad Prism, and EC50 or K_{D} values were calculated from the resulting curve fit.

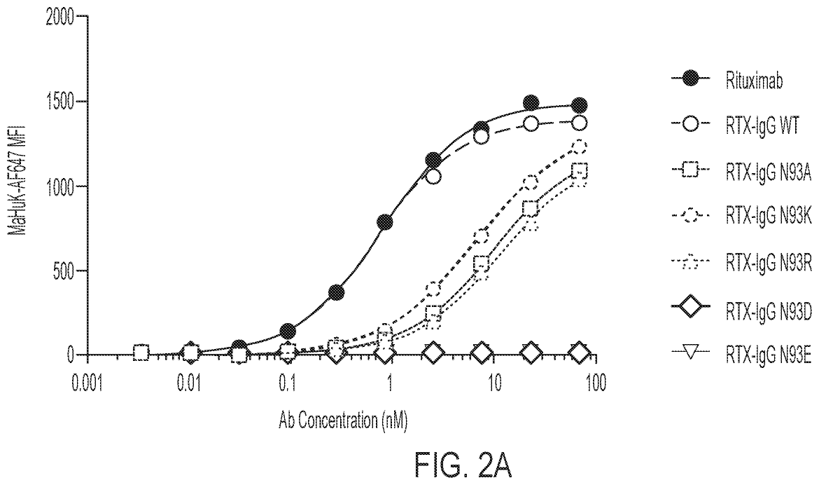

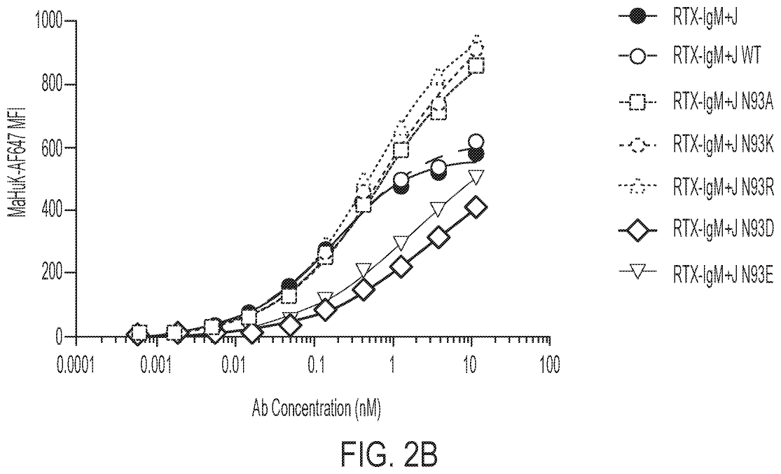

The results are shown in FIG. 2A (IgG) and FIG. 2B (IgM+J) and in Tables 4 and 5. The N93D and N93E mutants had no measurable affinity on Ramos as IgGs and exhibited about 10-fold higher EC50s (2.8 nM and 1.9 nM, respectively) as IgM's when compared to wt rituximab (0.16 nM or 0.19 nM for two different batches).

TABLE 4
EC50 Values for IgG Antibodies

| # | Ab | EC50 |
|---|---|---|
| 1 | Rituximab | 0.81 |
| 2 | RTX-IgG WT | 0.73 |
| 3 | RTX-IgG N93A | 9.8 |
| 4 | RTX-IgG N93K | 7.1 |
| 5 | RTX-IgG N93R | 12 |
| 6 | RTX-IgG N93D | ND |
| 7 | RTX-IgG N93E | ND |

TABLE 5
EC50 Values for IgM Antibodies

| # | Ab | EC50 |
|---|---|---|
| 1 | RTX-IgM + J | 0.16 |
| 2 | RTX-IgM + J WT | 0.19 |
| 3 | RTX-IgM + J N93A | 0.69 |
| 4 | RTX-IgM + J N93K | 0.88 |
| 5 | RTX-IgM + J N93R | 0.52 |
| 6 | RTX-IgM + J N93D | 2.8 |
| 7 | RTX-IgM + J N93E | 1.9 |

The N93D and N93E mutants showed reduced affinity for CD20 on Ramos cells as opposed to wild-type rituximab. In fact, the N93D and N93E mutants had no measurable affinity on Ramos as IgGs. As IgMs, the two mutants had roughly 10-fold higher EC50s than wild-type rituximab as IgM.

### Example 4: CDC Activity of IgG and IgM Anti-CD20 Mutants on Ramos and CA46 Cells

The ability of the IgG and IgM altered-affinity mutants produced in Example 1 to facilitate complement-dependent cytotoxicity on two B cell lines was evaluated by the following method. Two B cell lines with different CD20 densities as shown in Example 2, Ramos (higher density CD20) and CA46 (mid-to low density CD20) were maintained in culture in RPMI complete medium (RPMI-C; RPMI 1640 medium, 10 mM HEPES, 1 mM sodium pyruvate, 1×MEM non-essential amino acids, 1× GlutaMAX) containing 10-20% heat-inactivated fetal bovine serum (HI-FBS) (as appropriate for each cell line) at 37° C. in a 5% CO2 incubator. Split every 2-3 days as necessary to maintain the cell density between 0.2-2.0×10⁶ cells/mL. The cells were split every 2-3 days as necessary to maintain the cell density between 0.2-2.0×10⁶ cells/mL. About 5.0×10⁶ cells were recovered by centrifugation and were resuspended in 5.0 mL RPMI-C (1.0×10⁶ cells/mL). Purified anti-CD20 IgM and IgG antibodies, prepared as described in Example 1, were diluted to 30 μg/mL (IgG) or 3 μg/mL (IgM) in RPMI-C, 10% HI FBS, and were then serially diluted 3-fold in RPMI-C, 10% HI FBS in a non-tissue culture-treated 96-well plate (Falcon 351177). Cell samples (10 μL, about 10,000 cells/well) were combined with 10 μL of the serially diluted antibody samples in a 384-well plate (Thermo 164610) and were incubated for 2 h at 37° C. in a 5% CO2 incubator. Normal human serum complement (Quidel A113) was diluted to 30% in RPMI-C, 10% HI FBS (1.5 mL:3.5 mL), and 10 μL 30% NHS or RPMI-C, 10% HI FBS was added to each well. The plate was incubated for 4 h at 37° C. in a 5% CO2 incubator. Cell Titer-Glo reagent (10 μL, Promega G7572) was added to each well, mixed for two minutes on plate shaker, and then incubated for 10 minutes at RT. Luminescence was read on a PerkinElmer EnVision 2104. The raw data was imported into data into Excel and background was subtracted (signal from media+Cell Titer-Glo, no cells) from all values. The data was transferred to GraphPad Prism. The Y values were normalized such that 100% cell viability is defined as the averaged maximal value for cells incubated in the absence of antibody. The antibody concentrations (nM) were transformed using X=log(X), and then fitted to the data to a curve using a variable slope 4-parameter logistic fit [agonist vs. log(X)]. EC50 values were calculated from the resulting curve fit.

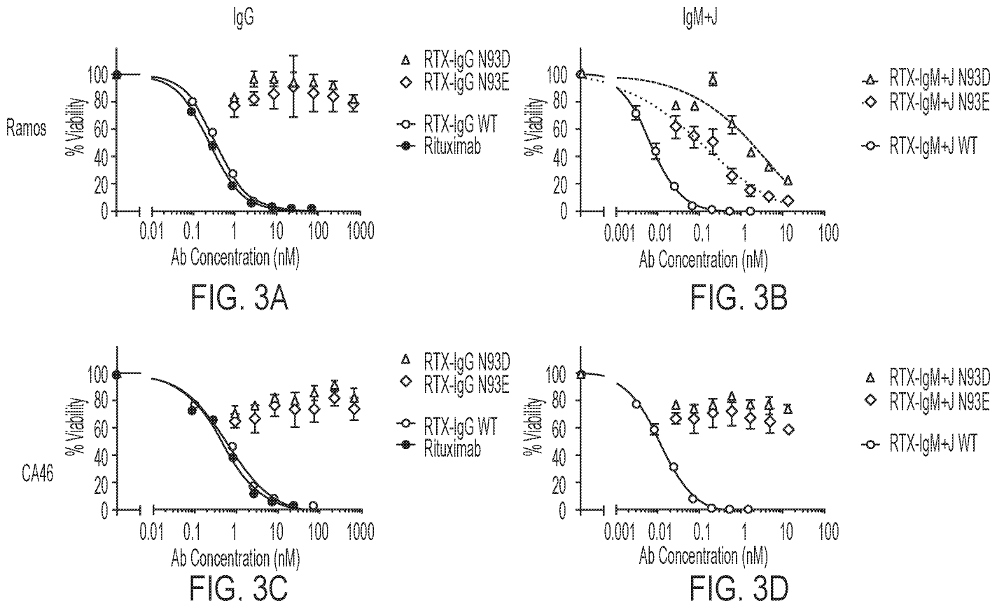

The results are shown in FIG. 3A-D. In the higher CD20 density Ramos cells, the N93D and N93E mutants showed no CDC activity whatsoever as IgGs (FIG. 3A), but given the increased avidity of IgM, the low affinity The N93D and N93E mutants showed measurable CDC activity (EC50 of about 0.1 to 10 nM) as high avidity IgMs (FIG. 3B). In contrast, in the lower CD20 density CA46 cells, neither the IgG (FIG. 3C) nor the IgM versions (FIG. 3D) of the N93D and N93E mutants showed measurable CDC activity.

### Example 5: TDCC Activity of IgM Anti-CD20 Mutants on Ramos and CA46 Cells

The ability of the IgM altered-affinity mutants produced in Example 1 to facilitate T cell-dependent cellular cytotoxicity (TDCC) on two B cell lines, and also on normal B cells from healthy donors, was evaluated by the following method.

One day prior to the experiment, CD8+ T cells (Precision for Medicine 84300) were thawed and resuspended at a cell density of approximately 1.0-2.0×10⁶ cells/mL in RPMI complete medium (RPMI-C; RPMI 1640 medium, 10 mM HEPES, 1 mM sodium pyruvate, 1×MEM non-essential amino acids, 1× GlutaMAX) containing 10% heat-inactivated fetal bovine serum (HI-FBS) and then rested overnight at 37° C. in a 5% CO2 incubator. On the day of the experiment, the T cells were adjusted to a density of approximately 1.0×10⁶ cells/mL in RPMI-C, 10% HI-FBS. Two B cell lines with different CD20 densities as shown in Example 2, Ramos (higher density CD20) and CA46 (mid- to low-density CD20), were maintained in culture in RPMI-C containing 10-20% HI-FBS (as appropriate for each cell line) at 37° C. in a 5% CO2 incubator. The cells were split every 2-3 days as necessary to maintain the cell density between 0.2-2.0×10⁶ cells/mL.

About 1.0×10⁶ B cells from healthy donors were recovered by centrifugation and were resuspended in 1.0 mL RPMI-C, 10% HI-FBS (1.0×106 cells/mL). The cells were fluorescently labeled by addition of CellTrace Oregon Green 488 (LifeTech 34550) to a final concentration of 2-5 μM followed by incubation for about 30 min at 37° C. in a 5% CO2 incubator. The labeled cells were washed twice with 10 mL of RPMI-C to remove excess label and resuspended at a final density of approximately 1.0×10⁵ cells/mL in RPMI-C. Fluorescent labeling was confirmed by examining the cells in a fluorescence microscope.

Purified, bispecific anti-CD20 IgM antibodies comprising a modified J-chain that specifically binds to CD3ε (J-chain amino acid sequence SEQ ID NO: 46), prepared as described in Example 1, were diluted to 2 μg/mL (wild type) or 20 μg/mL (variants) in RPMI-C, 10% HI FBS and were then serially diluted 3-fold in RPMI-C, 10% HI FBS in a non TC-treated 96-well U-bottom plate (Falcon 351177). Fluorescently-labeled B cells (50 μL, about 5,000 cells/well) were combined with 50 μL of the serially diluted antibody samples and CD8+ T cells (50 μL, about 50,000 cells/well) in a 96-well U-bottom plate (Falcon 351177) and incubated at 37° C. in a 5% CO₂ incubator. After about 48 h, 5 μL/well of CountBright Absolute Counting Beads (Invitrogen C36950) was added and the cells were washed once with 200 μL/well of FACS buffer (FBS; BD 554656) and then resuspended in about 60 μL/well FBS containing 1% 7-AAD (BD 51-88881E). The cell-bead mixture was analyzed by flow cytometry using a FACSCalibur flow cytometer (BD) and the data was saved in list mode. The data was analyzed using software program FlowJo, version 10 (BD). The B cells were gated on side scatter (SSC) vs. fluorescence in the FL1 (green) channel and live B cells were gated on FL1 fluorescence vs. non-fluorescence in the FL4 (red) channel. The counting beads were gated on SSC vs. FL1 fluorescence. The number of live B cells/well was normalized for losses due to washing using the counting beads. Percent lysis was calculated by dividing the normalized number of live cells/well in treated samples from the average normalized number of live cells/well in untreated samples, subtracting the resulting ratio from 1 and multiplying the result by 100. The data was further analyzed using software program Prism, version 7 (GraphPad). The antibody concentrations (pM) were transformed using X=log(X) and fitted to a curve using a variable slope 4-parameter logistic fit [agonist vs. log(X)]. The EC50 values were calculated from the curve fit.

The results are shown in FIG. 4A-4C, and in Table 6.

TABLE 6
TDCC Activity of IgM anti-CD20 Reduced Affinity Mutants

| | Ramos | | B cells | | CA46 | |
|---|---|---|---|---|---|---|
| Antibody | EC50 | % Max. | EC50 | % Max. | EC50 | % Max. |
| RTX-IgM V15J | 9 | 98 | 9 | 56 | 14 | 92 |
| (wt) | | | | | | |
| RTX-IgM V15J | 107 | 80 | 145 | 27 | NM | NM |
| (N93E) | | | | | | |
| RTX-IgM V15J | 722 | 60 | NM | NM | NM | NM |
| (N93D) | | | | | | |

In the higher CD20 density Ramos cells, the N93D and N93E IgM mutants showed measurable TDCC activity (EC50 of about 0.1 to 0.7 nM) as high avidity IgMs (FIG. 4C). In contrast, in the lower CD20 density CA46 cells, the N93D and N93E IgM mutants showed no measurable TDCC activity (FIG. 4A). The N93E mutant killed up to 27% of normal healthy B cells with a modest EC50 of 0.14 nM, where the N93D mutant showed no appreciable TDCC activity (FIG. 4B).

The breadth and scope of the present disclosure should not be limited by any of the above-described exemplary embodiments but should be defined only in accordance with the following claims and their equivalents.

TABLE 7
Additional Sequences

| SEQ ID | | |
|---|---|---|
| NO | Description | Sequence |
| 1 | Human IgM Constant | GSASAPTLFPLVSCENSPSDTSSVAVGCLAQDFLPDSITFSWKYKNNSDISSTRGFPSVLRGGKYAATSQVL |
| | region IMGT allele | LPSKDVMQGTDEHVVCKVQHPNGNKEKNVPLPVIAELPPKVSVFVPPRDGFFGNPRKSKLICQATGFSPRQ |
| | IGHM*03 | IQVSWLREGKQVGSGVTTDQVQAEAKESGPTTYKVTSTLTIKESDWLSQSMFTCRVDHRGLTFQQNASSM |
| | | CVPDQDTAIRVFAIPPSFASIFLTKSTKLTCLVTDLTTYDSVTISWTRQNGEAVKTHTNISESHPNATFSAVG |
| | | EASICEDDWNSGERFTCTVTHTDLPSPLKQTISRPKGVALHRPDVYLLPPAREQLNLRESATITCLVTGFSPA |
| | | DVFVQWMQRGQPLSPEKYVTSAPMPEPQAPGRYFAHSILTVSEEEWNTGETYTCVVAHEALPNRVTERTV |
| | | DKSTGKPTLYNVSLVMSDTAGTCY |
| 2 | Human IgM Constant | GSASAPTLFPLVSCENSPSDTSSVAVGCLAQDFLPDSITFSWKYKNNSDISSTRGFPSVLRGGKYAATSQVL |
| | region IMGT allele | LPSKDVMQGTDEHVVCKVQHPNGNKEKNVPLPVIAELPPKVSVFVPPRDGFFGNPRKSKLICQATGFSPRQ |
| | IGHM*04 | IQVSWLREGKQVGSGVTTDQVQAEAKESGPTTYKVTSTLTIKESDWLGQSMFTCRVDHRGLTFQQNASSM |
| | | CVPDQDTAIRVFAIPPSFASIFLTKSTKLTCLVTDLTTYDSVTISWTRQNGEAVKTHTNISESHPNATFSAVG |
| | | EASICEDDWNSGERFTCTVTHTDLPSPLKQTISRPKGVALHRPDVYLLPPAREQLNLRESATITCLVTGFSPA |
| | | DVFVQWMQRGQPLSPEKYVTSAPMPEPQAPGRYFAHSILTVSEEEWNTGETYTCVVAHEALPNRVTERTV |
| | | DKSTGKPTLYNVSLVMSDTAGTCY |
| 3 | Human IgA1 Constant | ASPTSPKVFPLSLCSTQPDGNVVIACLVQGFFPQEPLSVTWSESGQGVTARNFPPSQDASGDLYTTSSQLTLP |
| | Region | ATQCLAGKSVTCHVKHYTNPSQDVTVPCPVPSTPPTPSPSTPPTPSPSCCHPRLSLHRPALEDLLLGSEANLT |
| | | CTLTGLRDASGVTFTWTPSSGKSAVQGPPERDLCGCYSVSSVLPGCAEPWNHGKTFTCTAAYPESKTPLTA |
| | | TLSKSGNTFRPEVHLLPPPSEELALNELVTLTCLARGFSPKDVLVRWLQGSQELPREKYLTWASRQEPSQG |
| | | TTTFAVTSILRVAAEDWKKGDTFSCMVGHEALPLAFTQKTIDRLAGKPTHVNVSVVMAEVDGTCY |
| 4 | Human IgA2 Constant | ASPTSPKVFPLSLDSTPQDGNVVVACLVQGFFPQEPLSVTWSESGQNVTARNFPPSQDASGDLYTTSSQLTL |
| | Region | PATQCPDGKSVTCHVKHYTNPSQDVTVPCPVPPPPPCCHPRLSLHRPALEDLLLGSEANLTCTLTGLRDAS |
| | | GATFTWTPSSGKSAVQGPPERDLCGCYSVSSVLPGCAQPWNHGETFTCTAAHPELKTPLTANITKSGNTFR |
| | | PEVHLLPPPSEELALNELVTLTCLARGFSPKDVLVRWLQGSQELPREKYLTWASRQEPSQGTTTFAVTSILR |
| | | VAAEDWKKGDTFSCMVGHEALPLAFTQKTIDRLAGKPTHVNVSVVMAEVDGTCY |
| 5 | Precursor Human | MLLFVLTCLLAVFPAISTKSPIFGPEEVNSVEGNSVSITCYYPPTSVNRHTRKYWCRQGARGGCITLISSEGY |
| | Secretory Component | VSSKYAGRANLTNFPENGTFVVNIAQLSQDDSGRYKCGLGINSRGLSFDVSLEVSQGPGLLNDTKVYTVDL |
| | | GRTVTINCPFKTENAQKRKSLYKQIGLYPVLVIDSSGYVNPNYTGRIRLDIQGTGQLLFSVVINQLRLSDAG |
| | | QYLCQAGDDSNSNKKNADLQVLKPEPELVYEDLRGSVTFHCALGPEVANVAKFLCRQSSGENCDVVVNT |
| | | LGKRAPAFEGRILLNPQDKDGSFSVVITGLRKEDAGRYLCGAHSDGQLQEGSPIQAWQLFVNEESTIPRSPT |
| | | VVKGVAGGSVAVLCPYNRKESKSIKYWCLWEGAQNGRCPLLVDSEGWVKAQYEGRLSLLEEPGNGTFTV |
| | | ILNQLTSRDAGFYWCLTNGDTLWRTTVEIKIIEGEPNLKVPGNVTAVLGETLKVPCHFPCKFSSYEKYWCK |
| | | WNNTGCQALPSQDEGPSKAFVNCDENSRLVSLTLNLVTRADEGWYWCGVKQGHFYGETAAVYVAVEER |
| | | KAAGSRDVSLAKADAAPDEKVLDSGFREIENKAIQDPRLFAEEKAVADTRDQADGSRASVDSGSSEEQGG |
| | | SSRALVSTLVPLGLVLAVGAVAVGVARARHRKNVDRVSIRSYRTDISMSDFENSREFGANDNMGASSITQE |
| | | TSLGGKEEFVATTESTTETKEPKKAKRSSKEEAEMAYKDFLLQSSTVAAEAQDGPQEA |
| 6 | Precursor Human J | MKNHLLFWGVLAVFIKAVHVKAQEDERIVLVDNKCKCARITSRIIRSSEDPNEDIVERNIRIIVPLNNRENIS |
| | Chain | DPTSPLRTRFVYHLSDLCKKCDPTEVELDNQIVTATQSNICDEDSATETCYTYDRNKCYTAVVPLVYGGET |
| | | KMVETALTPDACYPD |
| 42 | Mature Human J | QEDERIVLVDNKCKCARITSRIIRSSEDPNEDIVERNIRIIVPLNNRENISDPTSPLRTRFVYHLSDLCKKCDPT |
| | Chain | EVELDNQIVTATQSNICDEDSATETCYTYDRNKCYTAVVPLVYGGETKMVETALTPDACYPD |
| 43 | Mature J* | QEDERIVLVDNKCKCARITSRIIRSSEDPNEDIVERNIRIIVPLNNRENISDPTSPLRTRFVYHLSDLCKKCDPT |
| | | EVELDNQIVTATQSNICDEDSATETCATYDRNKCYTAVVPLVYGGETKMVETALTPDACYPD |
| 44 | Visi VH | QVQLVQSGAEVKKPGASVKVSCKASGYTFISYTMHWVRQAPGQGLEWMGYINPRSGYTHYNQKLKDKA |
| | | TLTADKSASTAYMELSSLRSEDTAVYYCARSAYYDYDGFAYWGQGTLVTVSS |
| 45 | Visi VL | DIQMTQSPSSLSASVGDRVTITCSASSSVSYMNWYQQKPGKAPKRLIYDTSKLASGVPSRFSGSGSGTDFTL |
| | | TISSLQPEDFATYYCQQWSSNPPTFGGGTKLEIK |
| 46 | precursor modified | MGWSYIILFLVATATGVHSQVQLVQSGAEVKKPGASVKVSCKASGYTFISYTMHWVRQAPGQGLEWMG |
| | J-chain sequence | YINPRSGYTHYNQKLKDKATLTADKSASTAYMELSSLRSEDTAVYYCARSAYYDYDGFAYWGQGTLVTV |
| | for V15J | SSGGGGSGGGGSGGGGSDIQMTQSPSSLSASVGDRVTITCSASSSVSYMNWYQQKPGKAPKRLIYDTSKLA |
| | | SGVPSRFSGSGSGTDFTLTISSLQPEDFATYYCQQWSSNPPTFGGGTKLEIKGGGGSGGGGSGGGGSQEDER |
| | | IVLVDNKCKCARITSRIIRSSEDPNEDIVERNIRIIVPLNNRENISDPTSPLRTRFVYHLSDLCKKCDPTEVELD |
| | | NQIVTATQSNICDEDSATETCYTYDRNKCYTAVVPLVYGGETKMVETALTPDACYPD |
| 47 | precursor modified | MGWSYIILFLVATATGVHSQVQLVQSGAEVKKPGASVKVSCKASGYTFISYTMHWVRQAPGQGLEWMG |
| | J-chain sequence | YINPRSGYTHYNQKLKDKATLTADKSASTAYMELSSLRSEDTAVYYCARSAYYDYDGFAYWGQGTLVTV |
| | for V15J* | SSGGGGSGGGGSGGGGSDIQMTQSPSSLSASVGDRVTITCSASSSVSYMNWYQQKPGKAPKRLIYDTSKLA |
| | | SGVPSRFSGSGSGTDFTLTISSLQPEDFATYYCQQWSSNPPTFGGGTKLEIKGGGGSGGGGSGGGGSQEDER |
| | | IVLVDNKCKCARITSRIIRSSEDPNEDIVERNIRIIVPLNNRENISDPTSPLRTRFVYHLSDLCKKCDPTEVELD |
| | | NQIVTATQSNICDEDSATETCATYDRNKCYTAVVPLVYGGETKMVETALTPDACYPD |
| 48 | SP34 VH | EVQLVESGGGLVQPKGSLKLSCAASGFTFNTYAMNWVRQAPGKGLEWVARIRSKYNNYATYYADSVKD |
| | | RFTISRDDSQSILYLQMNNLKTEDTAMYYCVRHGNFGNSYVSWFAYWGQGTLVTVSS |
| 49 | SP34 VH CDR1 | GFTFNTYAMN |
| 50 | SP34 VH CDR2 | ARIRSKYNNYATYYADSVKD |
| 51 | SP34 VH CDR3 | VRHGNFGNSYVSWFAY |
| 52 | SP34 VL | QAVVTQESALTTSPGETVTLTCRSSTGAVTTSNYANWVQEKPDHLFTGLIGGTNKRAPGVPARFSGSLIGD |
| | | KAALTITGAQTEDEAIYFCALWYSNLWVFGGGTKLTVL |
| 53 | SP34 VL CDR1 | RSSTGAVTTSNYAN |
| 54 | SP34 VL CDR2 | GTNKRAP |
| 55 | SP34 VL CDR3 | ALWYSNL |
| 56 | SJ* | MGWSYIILFLVATATGVHSEVQLVESGGGLVQPKGSLKLSCAASGFTFNTYAMNWVRQAPGKGLEWVAR |
| | | IRSKYNNYATYYADSVKDRFTISRDDSQSILYLQMNNLKTEDTAMYYCVRHGNFGNSYVSWFAYWGQGT |
| | | LVTVSSGGGGSGGGGSGGGGSQAVVTQESALTTSPGETVTLTCRSSTGAVTTSNYANWVQEKPDHLFTGL |
| | | IGGTNKRAPGVPARFSGSLIGDKAALTITGAQTEDEAIYFCALWYSNLWVFGGGTKLTVLGGGGSGGGGS |
| | | GGGGSQEDERIVLVDNKCKCARITSRIIRSSEDPNEDIVERNIRIIVPLNNRENISDPTSPLRTRFVYHLSDLCK |
| | | KCDPTEVELDNQIVTATQSNICDEDSATETCATYDRNKCYTAVVPLVYGGETKMVETALTPDACYPD |
| 57 | 5-linker | GGGGS |
| 58 | 10-linker | GGGGSGGGGS |
| 59 | 15-linker | GGGGSGGGGSGGGGS |
| 60 | 20-linker | GGGGSGGGGSGGGGSGGGGS |
| 61 | 25-linker | GGGGSGGGGSGGGGSGGGGSGGGGS |

What is claimed is:

1. A multimeric binding molecule comprising two, five, or six bivalent binding units collectively comprising four, ten, or twelve binding unit-associated antigen binding domains, respectively;
wherein each binding unit comprises two antibody heavy chains, each comprising an IgA, IgA-like, IgM, or IgM-like heavy chain constant region or multimerizing fragment or variant thereof and a binding unit-associated antigen-binding domain or subunit thereof,
wherein at least three of the binding-unit-associated antigen-binding domains of the binding molecule specifically bind to the same predetermined target on the surface of a cell, and
wherein the binding molecule preferentially binds to a cell expressing the predetermined target at a higher density relative to a cell expressing the predetermined target at a lower density.

2. The binding molecule of claim 1, wherein each antibody heavy chain comprises a heavy chain variable region (VH) portion of a binding-unit-associated antigen-binding domain.

3. The binding molecule of claim 1 or claim 2, wherein the at least three binding unit-associated antigen-binding domains that bind to the same predetermined target on the surface of a cell each comprise a heavy chain variable region (VH) and light chain variable region (VL).

4. The binding molecule of any one of claims 1 to 3, wherein the at least three antigen-binding domains each bind to the same epitope on the predetermined target.

5. The binding molecule of claim 4, wherein the at least three binding-unit-associated antigen-binding domains that bind to the same predetermined target on the surface of a cell are identical.

6. The binding molecule of claim 5, wherein at least four, at least five, at least six, at least seven, at least eight, at least nine, at least ten, at least eleven, or twelve binding-unit-associated antigen-binding domains bind to the same predetermined target on the surface of a cell and are identical.

7. The binding molecule of claim 5 or claim 6, which preferentially binds to a cell expressing the predetermined target at a higher density relative to a reference bivalent IgG antibody having just two of the identical binding-unit-associated antigen-binding domains that specifically bind to the predetermined target on the surface of the cell.

8. The binding molecule of claim 7, which can bind to a cell expressing the target antigen at higher density, wherein the reference bivalent IgG antibody having just two of the identical binding-unit-associated antigen-binding domains that specifically bind to the predetermined target on the surface of the cell cannot bind to the cell expressing the target antigen at higher density.

9. The binding molecule of any one of claims 1 to 8, which does not detectably bind to a cell expressing the predetermined target at lower density.

10. The binding molecule of any one of claims 1 to 9, wherein the cell expressing the predetermined target at high density is a cancer cell and the cell expressing the predetermined target at low density is a normal cell.

11. The binding molecule of any one of claims 1 to 10, which is pentameric or hexameric, comprising five or six bivalent IgM or IgM-like binding units, respectively, wherein each binding unit comprises two IgM or IgM-like heavy chain constant regions or multimerizing fragments or variants thereof each associated with a binding unit-associated antigen-binding domain or subunit thereof.

12. The binding molecule of claim 11, wherein the IgM or IgM-like heavy chain constant regions or multimerizing fragments or variants thereof each comprise a Cμ4 domain and an IgM tailpiece (tp) domain.

13. The binding molecule of claim 11 or claim 12, wherein the IgM or IgM-like heavy chain constant regions or multimerizing fragments or variants thereof each further comprise a Cμ1 domain, a Cμ2 domain, a Cμ3 domain, or any combination thereof.

14. The binding molecule of any one of claims 11 to 13, wherein the IgM or IgM-like heavy chain constant regions comprise human IgM constant regions or human-derived IgM-like constant regions.

15. The binding molecule of any one of claims 11 to 14, wherein each binding unit comprises two IgM or IgM-like heavy chains each comprising a VH situated amino terminal to the IgM or IgM-like constant region or multimerizing fragment or variant thereof, and two immunoglobulin light chains each comprising a VL situated amino terminal to an immunoglobulin light chain constant region.

16. The binding molecule of any one of claims 11 to 15, which is pentameric, and further comprises a J-chain, or functional fragment or variant thereof.

17. The binding molecule of any one of claims 1 to 10, which is dimeric and comprises two bivalent IgA or IgA-like binding units and a J-chain or a functional fragment or variant thereof, wherein each binding unit comprises two IgA or IgA-like heavy chain constant regions or multimerizing fragments or variants thereof each associated with a binding unit-associated antigen-binding domain or subunit thereof.

18. The binding molecule of claim 17, further comprising a secretory component, or functional fragment or variant thereof.

19. The binding molecule of claim 17 or claim 18, wherein the IgA or IgA-like heavy chain constant regions or multimerizing fragments or variants thereof each comprise a Cα3 domain and an IgA tailpiece (tp) domain.

20. The binding molecule of claim 19, wherein the IgA or IgA-like heavy chain constant regions or multimerizing fragments or variants thereof each further comprise a Cα1 domain, a Cα2 domain, an IgA hinge region, or any combination thereof.

21. The binding molecule of any one of claims 17 to 20, wherein the IgA or IgA-like heavy chain constant regions or multimerizing fragments or variants thereof are human IgA heavy chain constant regions or human-derived IgA-like heavy chain constant regions.

22. The binding molecule of any one of claims 17 to 21, wherein each binding unit comprises two IgA or IgA-like heavy chains each comprising a VH situated amino terminal to the IgA or IgA-like heavy chain constant region or multimerizing fragment or variant thereof, and two immunoglobulin light chains each comprising a VL situated amino terminal to an immunoglobulin light chain constant region.

23. The binding molecule of any one of claims 16 to 22, wherein the J-chain or fragment or variant thereof is a human J-chain comprising the amino acid sequence SEQ ID NO: 42 or a functional fragment or variant thereof.

24. The binding molecule of any one of claims 16 to 23, wherein the J-chain or functional fragment or variant thereof is a variant J-chain comprising one or more single amino acid substitutions, deletions, or insertions relative to a wild-type J-chain, which can affect serum half-life of the binding molecule; and wherein the binding molecule exhibits an increased serum half-life upon administration to an animal relative to a reference binding molecule that is identical except for the one or more single amino acid substitutions, deletions, or insertions, and is administered in the same way to the same animal species.

25. The binding molecule of claim 24, wherein the J-chain or functional fragment or variant thereof comprises an amino acid substitution at the amino acid position corresponding to amino acid Y102 of the wild-type human J-chain (SEQ ID NO: 42).

26. The binding molecule of claim 25, wherein the amino acid corresponding to Y102 of SEQ ID NO: 42 is substituted with alanine (A), serine (S), or arginine (R).

27. The binding molecule of claim 26, wherein the amino acid corresponding to Y102 of SEQ ID NO: 42 is substituted with alanine (A).

28. The binding molecule of claim 27, wherein the J-chain is a variant human J-chain and comprises the amino acid sequence SEQ ID NO: 43 (J*).

29. The binding molecule of any one of claims 16 to 28, wherein the J-chain or fragment or variant thereof is a modified J-chain further comprising a heterologous moiety, wherein the heterologous moiety is chemically conjugated or fused to the J-chain or fragment or variant thereof.

30. The binding molecule of claim 29, wherein the heterologous moiety is a heterologous polypeptide.

31. The binding molecule of claim 30, wherein the heterologous polypeptide is fused to the J-chain or fragment or variant thereof via a peptide linker.

32. The binding molecule of claim 31, wherein the peptide linker consists of SEQ ID NO: 57, SEQ ID NO: 58, SEQ ID NO: 59, SEQ ID NO: 60, or SEQ ID NO: 61.

33. The binding molecule of any one of claims 30 to 32, wherein the heterologous polypeptide is fused to the N-terminus of the J-chain or fragment or variant thereof, to the C-terminus of the J-chain or fragment or variant thereof, or wherein heterologous polypeptides are fused to both the N- and C-terminus of the J-chain or fragment or variant thereof.

34. The binding molecule of any one of claims 30 to 33, comprising at least two heterologous polypeptides fused to the J-chain or fragment or variant thereof, wherein the at least two heterologous polypeptides can be the same or different.

35. The binding molecule of any one of claims 30 to 34, wherein at least one heterologous polypeptide comprises a J-chain-associated antigen-binding domain.

36. The binding molecule of claim 35, wherein the J-chain-associated antigen-binding domain is an antibody or antigen-binding fragment thereof.

37. The binding molecule of claim 36, wherein the antigen-binding fragment comprises an Fab fragment, an Fab′ fragment, an F(ab′)2 fragment, an Fd fragment, an Fv fragment, a single-chain Fv (scFv) fragment, a disulfide-linked Fv (sdFv) fragment, or any combination thereof.

38. The binding molecule of claim 37, wherein the J-chain-associated antigen-binding domain comprises a scFv fragment.

39. The binding molecule of any one of claims 35 to 38, wherein the J-chain-associated antigen-binding domain specifically binds to an immune effector cell.

40. The binding molecule of claim 39, wherein the immune effector cell is a T cell or an NK cell.

41. The binding molecule of claim 39 or claim 40, wherein the immune effector cell is a T cell, and wherein the J-chain-associated antigen-binding domain comprises a scFv fragment that specifically binds to CD3R.

42. The binding molecule of claim 41, wherein the T cell is a CD8+ cytotoxic T cell.

43. The binding molecule of claim 41 or claim 42, wherein the scFv fragment comprises a heavy chain variable region (VH) and a light chain variable region (VL), wherein the VH comprises the VH complementarity-determining regions VHCDR1, VHCDR2, and VHCDR3 comprising the amino acid sequences SEQ ID NO: 49, SEQ ID NO: 50, and SEQ ID NO: 51, respectively, or SEQ ID NO: 49, SEQ ID NO: 50, and SEQ ID NO: 51 with one, two, or three amino acid substitutions in one or more of the VHCDRs, and wherein the VL comprises the VL complementarity-determining regions VLCDR1, VLCDR2, and VLCDR3 comprising the amino acid sequences SEQ ID NO: 53, SEQ ID NO: 54, and SEQ ID NO: 55, respectively, or SEQ ID NO: 53, SEQ ID NO: 54, and SEQ ID NO: 55 with one, two, or three amino acid substitutions in one or more of the VLCDRs.

44. The binding molecule of claim 43, wherein the scFv fragment comprises the VH amino acid sequence SEQ ID NO: 48 and the VL amino acid sequence SEQ ID NO: 52.

45. The binding molecule of claim 41 or claim 42, wherein the scFv fragment comprises a heavy chain variable region (VH) and a light chain variable region (VL), wherein the VH and VL comprise the amino acid sequences SEQ ID NO: 44 and SEQ ID NO: 45, respectively.

46. The binding molecule of claim 41 or claim 42, wherein the modified J-chain comprises amino acids 20 to 412 of SEQ ID NO: 46 (V15J), amino acids 20 to 412 of SEQ ID NO: 47 (V15J*), or amino acids 20 to 420 of SEQ ID NO: 56 (SJ*).

47. The binding molecule of claim 40, wherein the immune effector cell is an NK cell, and wherein the scFv fragment specifically binds to CD16.

48. The binding molecule of any one of claims 29 to 47, wherein the modified J-chain further comprises an immune stimulatory agent (“ISA”) fused or chemically conjugated to the J-chain or fragment or variant thereof.

49. The binding molecule of claim 48, wherein the ISA comprises a cytokine or receptor-binding fragment or variant thereof.

50. The binding molecule of claim 49, wherein the ISA comprises (a) an interleukin-15 (IL-15) protein or receptor-binding fragment or variant thereof (“I”), and (b) an interleukin-15 receptor-α (IL-15Rα) fragment comprising the sushi domain or a variant thereof capable of associating with I (“R”), wherein the J-chain or fragment or variant thereof and at least one of I and R are associated as a fusion protein, and wherein I and R can associate to function as the ISA.

51. The binding molecule of any one of claims 48 to 50, wherein the ISA is fused to the J-chain via a peptide linker.

52. The binding molecule of any one of claims 1 to 51, wherein the predetermined target is B-cell maturation antigen (BCMA), CD19, CD20, EGFR, HER2 (ErbB2), ErbB3, ErbB4, CTLA4, PD-1, PD-L1, VEGF, VEGFR1, VEGFR2, CD52, CD30, prostate-specific membrane antigen (PSMA), CD38, GD2, SLAMF7, platelet-derived growth factor receptor A (PDGFRA), CD22, FLT3 (CD135), CD123, MUC-16, carcinoembryonic antigen-related cell adhesion molecule 1 (CEACAM-1), mesothelin, tumor-associated calcium signal transducer 2 (Trop-2), glypican-3 (GPC-3), human blood group H type 1 trisaccharide (Globo-H), sialyl Tn antigen (STn antigen), or CD33.

53. The binding molecule of any one of claims 5 to 52, wherein the at least three identical binding unit-associated antigen-binding domains specific for the predetermined target are reduced-affinity variants of an antigen-binding domain of an existing antibody known to bind to the predetermined target, wherein the antigen-binding domain of an existing antibody comprises a heavy chain variable region (VH) and light chain variable region (VL).

54. The binding molecule of claim 53, wherein the existing antibody is alemtuzumab, atezolizumab, atezolizumab, avelumab, bevacizumab, blinatumomab, brentuximab, capromab, cetuximab, daratumumab, denosumab, dinutuximab, durvalumab, elotuzumab, gemtuzumab, ibritumomab, ipilimumab, inotuzumab, necitumumab, nivolumab, nivolumab, obinutuzumab, ocrelizumab, ofatumumab, olaratumab, omalizumab, panitumumab, pembrolizumab, pertuzumab, ramucirumab, ranibizumab, rituximab, trastuzumab, or tremelimumab.

55. The binding molecule of claim 53 or claim 54, which binds to the predetermined target with a binding affinity for the predetermined target at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 500-fold, or at least 1000-fold lower than the existing antibody.

56. The binding molecule of any one of claims 53 to 55, wherein VH and VL of the antigen-binding domain of the existing antibody comprise, respectively, the amino acid sequences SEQ ID NO: 7 and SEQ ID NO: 8, SEQ ID NO: 14 and SEQ ID NO: 15, SEQ ID NO: 16 and SEQ ID NO: 17, SEQ ID NO: 18 and SEQ ID NO: 19, SEQ ID NO: 20 and SEQ ID NO: 21, SEQ ID NO: 22 and SEQ ID NO: 23, SEQ ID NO: 24 and SEQ ID NO: 25, SEQ ID NO: 26 and SEQ ID NO: 27, SEQ ID NO: 28 and SEQ ID NO: 29, SEQ ID NO: 30 and SEQ ID NO: 31, SEQ ID NO: 32 and SEQ ID NO: 33, SEQ ID NO: 34 and SEQ ID NO: 35, SEQ ID NO: 36 and SEQ ID NO: 37, SEQ ID NO: 38 and SEQ ID NO: 39, or SEQ ID NO: 40 and SEQ ID NO: 41.

57. The binding molecule of claim 56, wherein the antigen-binding domain of the existing antibody comprises the VH amino acid sequence SEQ ID NO: 7 and the VL amino acid sequence SEQ ID NO: 8, and wherein the reduced-affinity variant comprises an amino acid substitution at position N93 in the VL, SEQ ID NO: 8.

58. The binding molecule of claim 57, wherein the amino acid substitution is N93D or N93E.

59. The binding molecule of claim 58, wherein the at least three identical binding unit-associated antigen-binding domains specific for the predetermined target comprise the VH amino acid sequence SEQ ID NO: 7 and the VL amino acid sequence SEQ ID NO: 9 or SEQ ID NO: 10.

60. The binding molecule of any one of claims 57 to 59, which can direct complement-directed cytotoxicity (CDC) of B cells expressing CD20 at high density at an EC50 concentration at least 2-fold, at least 5-fold, at least 10-fold, at least 50-fold, at least 100-fold, at least 500-fold or at least 1000-fold lower than the EC50 concentration for B cells expressing CD20 at low density.

61. The binding molecule of any one of claims 57 to 60, which can direct CDC of B cells expressing CD20 at high density, but not B cells expressing CD20 at low density.

62. The binding molecule of any one of claims 57 to 61, which is a pentameric IgM or IgM-like binding molecule or a dimeric IgA or IgA-like binding molecule, and further comprises a modified J-chain comprising a scFv that specifically binds to CD3ε.

63. The binding molecule of claim 62, wherein the modified J-chain comprises amino acids 20 to 412 of SEQ ID NO: 46 (V15J), amino acids 20 to 412 of SEQ ID NO: 47 (V15J*), or amino acids 20 to 420 of SEQ ID NO: 56 (SJ*).

64. The binding molecule of claim 62 or claim 63, which can direct T-cell directed cellular cytotoxicity (TDCC) or both TDCC and CDC of B cells expressing CD20 at high density at an EC50 concentration at least 2-fold, at least 5-fold, at least 10-fold, at least 50-fold, at least 100-fold, at least 500-fold or at least 1000-fold lower than the EC50 concentration for a B cell line or normal B cells, expressing CD20 at low density.

65. The binding molecule of any one of claims 62 to 64, which can direct TDCC or both TDCC and CDC of B cells expressing CD20 at high density, but not B cells expressing CD20 at low density.

66. The binding molecule of any one of claims 60 to 65, wherein the B cells expressing CD20 at both high and low density are lymphoma cell lines.

67. The binding molecule of claim 66, wherein the lymphoma cell line expressing CD20 at high density is a Ramos cell line, a Raji cell line, a DoHH-2 cell line, a JeKo-1 cell line, a Z-138 cell line, a Daudi cell line, a Granta cell line, or a DoHH2 cell line, and the lymphoma cell line expressing CD20 at low density is a CA46 cell line, a Nalm-1 cell line, a Toledo cell line, a BJAB cell line, a Kasumi-2 cell line, an RPMI 8226 cell line, an HT cell line, an SU-DHL-8 cell line, a JM1 cell line, a Namalwa cell line, a Nalm-6 cell line, or a Z138 cell line.

68. The binding molecule of any one of claim 66 or claim 67, which can direct CDC of Ramos cells, wherein an equivalent amount of a monospecific bivalent IgG1 antibody comprising the same antigen-binding domains as the binding molecule shows no detectable complement-mediated killing of Ramos cells.

69. The binding molecule of any one of claims 60 to 65, wherein the B cells expressing CD20 at high density are CD20-positive malignant B cells in a subject with cancer, and the B cells expressing CD20 at low density are normal B cells in the subject with cancer.

70. The binding molecule of claim 69, wherein the cancer is a CD20-positive leukemia, lymphoma, or myeloma.

71. The binding molecule of claim 69 or claim 70, wherein the subject is human.

72. A composition comprising the binding molecule of any one of claims 1 to 71, and a carrier.

73. An isolated polynucleotide comprising a nucleic acid molecule that encodes the binding molecule of any one of claims 1 to 71, or an antigen-binding and multimerizing fragment thereof, or a subunit thereof.

74. The polynucleotide of claim 73, wherein the subunit is an antibody heavy chain or multimerizing fragment or variant thereof, an antibody light chain or fragment thereof, a J-chain or fragment or variant thereof, or any combination thereof.

75. A vector comprising the polynucleotide of claim 73 or claim 74.

76. A host cell comprising the vector of claim 75.

77. A method for producing the binding molecule of any one of claims 1 to 71, comprising culturing the host cell of claim 76, and recovering the binding molecule.

78. An IgM or IgM-like antibody comprising five bivalent binding units and a modified J-chain,
wherein each binding unit comprises two human IgM or human-derived IgM-like heavy chain constant regions or multimerizing fragments or variants thereof, each associated with a binding unit-associated antigen-binding domain,
wherein each binding unit-associated antigen-binding domain of the antibody comprises the VH amino acid sequence SEQ ID NO: 7 and the VL amino acid sequence SEQ ID NO: 9 or SEQ ID NO: 10,
wherein the modified J-chain comprises a J-chain-associated binding domain comprising a scFv that specifically binds to CD3ε, and
wherein the IgM antibody can direct TDCC, CDC, or both TDCC and CDC of B cells expressing CD20 at high density at an EC50 concentration at least 2-fold, at least 5-fold, at least 10-fold, at least 50-fold, at least 100-fold, at least 500-fold or at least 1000-fold lower than the EC50 concentration for a B cell line or normal B cells, expressing CD20 at low density.

79. The IgM or IgM-like antibody of claim 78, which can direct CDC, TDCC, or both TDCC and CDC of B cells expressing CD20 at high density, but not B cells expressing CD20 at low density.

80. The IgM or IgM-like antibody of claim 78 or claim 79, wherein the modified J-chain comprises amino acids 20 to 412 of SEQ ID NO: 46 (V15J), amino acids 20 to 412 of SEQ ID NO: 47 (V15J*), or amino acids 20 to 420 of SEQ ID NO: 56 (SJ*).

81. The IgM or IgM-like antibody of any one of claims 78 to 80, wherein the high and low density CD20-expressing cells are lymphoma cell lines.

82. The IgM or IgM-like antibody of claim 81, wherein the cell expressing CD20 at high density is a Ramos cell line, and the cell expressing CD20 at low density is a CA46 cell line.

83. The IgM or IgM-like antibody of any one of claims 78 to 82, which can direct complement-mediated killing of Ramos cells, wherein an equivalent amount of a monospecific bivalent IgG1 antibody comprising the same antigen-binding domains shows no detectable complement-mediated killing of Ramos cells.

84. The IgM or IgM-like antibody of any one of claims 78 to 80, wherein the cell expressing CD20 at high density is a malignant B cell in a subject with cancer, and wherein the cell expressing CD20 at low density is a normal B cell in the subject with cancer.

85. The IgM or IgM-like antibody of claim 84, wherein the cancer is a CD20-positive leukemia, lymphoma, or myeloma.

86. The IgM or IgM-like antibody of claim 84 or claim 85, wherein the subject is human.

87. A method for treating cancer in a subject, comprising administering an effective amount of the binding molecule of any one of claims 1 to 71, the composition of claim 72, or the IgM or IgM-like antibody of any one of claims 78 to 86 to a subject in need of treatment.

88. Use of an effective amount of the binding molecule of any one of claims 1 to 71, the composition of claim 72, or the IgM or IgM-like antibody of any one of claims 78 to 86 in the preparation of a medicament for treating cancer.

89. The binding molecule of any one of claims 1 to 71, the composition of claim 72, or the IgM or IgM-like antibody of any one of claims 78 to 86, for use in treating cancer.
